(19) United States
(12) Patent Application Publication
(10) Pub. No.: US 2026/0191818 A1
(43) Pub. Date: Jul. 9, 2026

(54) TREATMENT REGIMENS AND METHODS OF USING QUERCETIN TO PREVENT AND/OR TREAT MUSCLE WASTING DISORDERS

(71) Applicant: University of South Carolina, Columbia, SC (US)

(72) Inventors: Elizabeth Murphy, Columbia, SC (US); Kandy Velazquez, Columbia, SC (US); Brandon VanderVeen, Columbia, SC (US); Daping Fan, Columbia, SC (US)

(73) Assignee: University of South Carolina, Columbia, SC (US)

(21) Appl. No.: 19/132,659

(22) PCT Filed: Oct. 26, 2023
(86) PCT No.: PCT/US2023/077827

§ 371 Date: May 23, 2025

Related U.S. Application Data
(60) Provisional application No. 63/427,486, filed on Nov. 23, 2022.

(51) Int. Cl. A61K 31/352; A61K 31/513; A61P 3/00; A61P 21/00

(52) U.S. Cl. CPC A61K 31/352; A61K 31/513; A61P 3/00; A61P 21/00

(57) ABSTRACT

Described herein are treatment regimens, therapies, pharmaceutical formulations and methods of using quercetin to prevent, treat, and/or protect from muscle wasting including 1) sarcopenia associated with aging; and 2) cachexia associated with chronic diseases and their treatments including but not limited to cancer, cardiovascular disease, chronic obstructive pulmonary disease (COPD), HIV, and kidney disease.

### STATEMENT REGARDING FEDERALLY SPONSORED RESEARCH

This invention was made with government support under NIH/NCCIH R43 AT011171. The government has certain rights in the invention.

### TECHNICAL FIELD

The subject matter disclosed herein is generally directed to treatment regimens, therapies, pharmaceutical formulations and methods of using quercetin to protect from muscle wasting including 1) sarcopenia associated with aging and 2) cachexia associated with chronic diseases and their treatments including but not limited to cancer, cardiovascular disease, chronic obstructive pulmonary disease (COPD), and kidney disease.

### BACKGROUND

Cachexia, the unintentional loss of body weight, is prevalent in chronic diseases and associated treatments including cancer, COPD, and kidney disease. For example, in cancer and cancer treatment, cachexia is associated with reduced physical function, decreased tolerance for treatment, and increased mortality. Moreover, a Cachexia diagnosis is consistent with a doubling in duration of hospital stay and an additional $4,000/case compared to non-cachectic patients. Similarly, sarcopenia, the loss of muscle tissue, strength, and function as part of the aging process, can lead to other health problems and complications including increasing the risk for falls and fractures and increasing the need for and length of hospitalizations and even death. Mitochondria are known to play a dominant role in driving Cachexia and sarcopenia. However, currently there are no FDA approved therapies to prevent or treat Cachexia or sarcopenia.

Accordingly, it is an object of the present disclosure to provide treatment regimens, therapies, and methods of using dietary quercetin to protect from muscle wasting associated with Cachexia and sarcopenia.

Citation or identification of any document in this application is not an admission that such a document is available as prior art to the present disclosure.

### SUMMARY

The above objectives are accomplished according to the present disclosure by providing in one embodiment, a method of treatment for protecting against muscle wasting induced muscle mass loss. The method may include administering a therapeutically effective amount of at least one polyphenol flavonoid to a subject, administration of the at least one polyphenol flavonoid protects against muscle wasting via increasing mitochondrial biogenesis, and administering the at least one polyphenol flavonoid increases muscle mass and muscle cross section in the subject. Further, administration of the at least one polyphenol flavonoid may prevent or treat Cachexia or sarcopenia. Yet again, administration of the at least one polyphenol flavonoid may prevent or treat Cachexia induced by chronic obstructive pulmonary disease, heart failure, chronic kidney disease, at least one cancer, chemotherapy, or combinations of the above. Still further, a subject undergoing the subject undergoing administration of the at least one polyphenol flavonoid may have increased expression of SEQ ID NO: 1 and reduced expression of SEQ ID NO: 2 in the subject as compared to prior to treatment with the method. Still further, the at least one polyphenol flavonoid may comprise 3,4,5,7-pentahydroxylflavone (quercetin). Moreover, administration of the at least one polyphenol flavonoid may equilibrate molecular signaling to protect against mitochondrial dysfunction. Yet again, administration of the at least one polyphenol flavonoid may increase expression of SEQ ID NO: 18 and decrease expression of SEQ ID NO: 6 and SEQ ID NO: 7 in the subject as compared to prior to treatment with the method. Furthermore, administration of the at least one polyphenol flavonoid may produce anti-cachectic properties demonstrated by preserving muscle mass and muscle cross sectional area with improved mitochondrial content in the subject. Still again, administration of the at least one polyphenol flavonoid may increase a number of intermyofibrillar mitochondria and may increase size of the intermyofibrillar mitochondria. Still yet further, administration of the at least one polyphenol flavonoid may be provided to a subject bearing at least one tumor. Again further, administration of the at least one polyphenol flavonoid may be administered with 5-fluorouracil. Further still, administration of the at least one polyphenol flavonoid may not reduce the tumor suppressive capability of 5-fluorouracil. Still moreover, administration of the at least one polyphenol flavonoid may increase succinate dehydrogenase activity in at least one myofibrillar fiber. Still yet, administration of the at least one polyphenol flavonoid may increase expression of SEQ ID NO: 19 and increase expression of SEQ ID NO: 20 in the subject as compared to prior to treatment with the method. Yet further, administration of the at least one polyphenol flavonoid may increase expression of SEQ ID NO: 17 and increase expression of SEQ ID NO: 18 in the subject as compared to prior to treatment with the method. Moreover, administration of the at least one polyphenol flavonoid may reduce expression of SEQ ID NO: 6 and reduce expression of SEQ ID NO: 7 in the subject as compared to prior to treatment with the method. Still yet further, administration of the at least one polyphenol flavonoid may equilibrate molecular signaling to protect against mitochondrial dysfunction.

These and other aspects, objects, features, and advantages of the example embodiments will become apparent to those having ordinary skill in the art upon consideration of the following detailed description of example embodiments.

### BRIEF DESCRIPTION OF THE DRAWINGS

An understanding of the features and advantages of the present disclosure will be obtained by reference to the following detailed description that sets forth illustrative embodiments, in which the principles of the disclosure may be utilized, and the accompanying drawings of which:

FIG. 1 shows graphs of quercetin's impact on body and tissue weights.

FIG. 2 shows histological images and graph of quercetin increasing muscle cross sectional area and succinate dehydrogenase activity.

FIG. 3 shows electron microscopy images and graphs showing quercetin increased mitochondrial size and number.

FIG. 4 shows a western blot and graphs showing quercetin increased mitochondrial content proteins.

FIG. 5 shows a western blot and graphs showing quercetin has modest impacts on general autophagy and mitophagy proteins.

FIG. 6 shows a western blot and graphs showing quercetin normalizes mitochondrial dynamics proteins.

FIG. 7 shows a western blot and graphs illustrating quercetin has modest impact of circulating IL-6 (SEQ ID NO: 9) and muscle inflammatory signaling.

FIG. 8 shows Table 1—a listing of used antibodies and probes.

The figures herein are for illustrative purposes only and are not necessarily drawn to scale.

### DETAILED DESCRIPTION OF THE EXAMPLE EMBODIMENTS

Before the present disclosure is described in greater detail, it is to be understood that this disclosure is not limited to particular embodiments described, and as such may, of course, vary. It is also to be understood that the terminology used herein is for the purpose of describing particular embodiments only and is not intended to be limiting.

Unless specifically stated, terms and phrases used in this document, and variations thereof, unless otherwise expressly stated, should be construed as open ended as opposed to limiting. Likewise, a group of items linked with the conjunction “and” should not be read as requiring that each and every one of those items be present in the grouping, but rather should be read as “and/or” unless expressly stated otherwise. Similarly, a group of items linked with the conjunction “or” should not be read as requiring mutual exclusivity among that group, but rather should also be read as “and/or” unless expressly stated otherwise.

Furthermore, although items, elements or components of the disclosure may be described or claimed in the singular, the plural is contemplated to be within the scope thereof unless limitation to the singular is explicitly stated. The presence of broadening words and phrases such as “one or more,” “at least,” “but not limited to” or other like phrases in some instances shall not be read to mean that the narrower case is intended or required in instances where such broadening phrases may be absent.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are cited to disclose and describe the methods and/or materials in connection with which the publications are cited. All such publications and patents are herein incorporated by references as if each individual publication or patent were specifically and individually indicated to be incorporated by reference. Such incorporation by reference is expressly limited to the methods and/or materials described in the cited publications and patents and does not extend to any lexicographical definitions from the cited publications and patents. Any lexicographical definition in the publications and patents cited that is not also expressly repeated in the instant application should not be treated as such and should not be read as defining any terms appearing in the accompanying claims. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Where a range is expressed, a further embodiment includes from the one particular value and/or to the other particular value. The recitation of numerical ranges by endpoints includes all numbers and fractions subsumed within the respective ranges, as well as the recited endpoints. Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less' and should be interpreted to include the specific ranges of ‘about x’, ‘about y’, and ‘about z’ as well as the ranges of ‘less than x’, less than y’, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y’, and ‘greater than z’. In addition, the phrase “about ‘x’ to ‘y’”, where ‘x’ and ‘y’ are numerical values, includes “about ‘x’ to about ‘y’”.

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, the singular forms “a”, “an”, and “the” include both singular and plural referents unless the context clearly dictates otherwise.

As used herein, “about,” “approximately,” “substantially,” and the like, when used in connection with a measurable variable such as a parameter, an amount, a temporal duration, and the like, are meant to encompass variations of and from the specified value including those within experimental error (which can be determined by e.g. given data set, art accepted standard, and/or with e.g. a given confidence interval (e.g. 90%, 95%, or more confidence interval from the mean), such as variations of +/−10% or less, +/−5% or less, +/−1% or less, and +/−0.1% or less of and from the specified value, insofar such variations are appropriate to perform in the disclosure. As used herein, the terms “about,” “approximate,” “at or about,” and “substantially” can mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

As used herein, a “biological sample” may contain whole cells and/or live cells and/or cell debris. The biological sample may contain (or be derived from) a “bodily fluid”. The present disclosure encompasses embodiments wherein the bodily fluid is selected from amniotic fluid, aqueous humour, vitreous humour, bile, blood serum, blood plasma, breast milk, cerebrospinal fluid, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof. Biological samples include cell cultures, bodily fluids, and cell cultures from bodily fluids. Bodily fluids may be obtained from a mammal organism, for example by puncture, or other collecting or sampling procedures.

As used herein, “agent” refers to any substance, compound, molecule, and the like, which can be administered to a subject on a subject to which it is administered to. An agent can be inert. An agent can be an active agent. An agent can be a primary active agent, or in other words, the component(s) of a composition to which the whole or part of the effect of the composition is attributed. An agent can be a secondary agent, or in other words, the component(s) of a composition to which an additional part and/or other effect of the composition is attributed.

As used herein, “active agent” or “active ingredient” refers to a substance, compound, or molecule, which is biologically active or otherwise that induces a biological or physiological effect on a subject to which it is administered to. In other words, “active agent” or “active ingredient” refers to a component or components of a composition to which the whole or part of the effect of the composition is attributed.

As used herein, “administering” refers to any suitable administration for the agent(s) being delivered and/or subject receiving said agent(s) and can be oral, topical, intravenous, subcutaneous, transcutaneous, transdermal, intramuscular, intra-joint, parenteral, intra-arteriole, intradermal, intraventricular, intraosseous, intraocular, intracranial, intraperitoneal, intralesional, intranasal, intracardiac, intraarticular, intracavernous, intrathecal, intravireal, intracerebral, and intracerebroventricular, intratympanic, intracochlear, rectal, vaginal, by inhalation, by catheters, stents or via an implanted reservoir or other device that administers, either actively or passively (e.g. by diffusion) a composition to the perivascular space and adventitia. For example, a medical device such as a stent can contain a composition or formulation disposed on its surface, which can then dissolve or be otherwise distributed to the surrounding tissue and cells. The term “parenteral” can include subcutaneous, intravenous, intramuscular, intra-articular, intra-synovial, intrasternal, intrathecal, intrahepatic, intralesional, and intracranial injections or infusion techniques. Administration routes can be, for instance, auricular (otic), buccal, conjunctival, cutaneous, dental, electro-osmosis, endocervical, endosinusial, endotracheal, enteral, epidural, extra-amniotic, extracorporeal, hemodialysis, infiltration, interstitial, intra-abdominal, intra-amniotic, intra-arterial, intra-articular, intrabiliary, intrabronchial, intrabursal, intracardiac, intracartilaginous, intracaudal, intracavernous, intracavitary, intracerebral, intracisternal, intracorneal, intracoronal (dental), intracoronary, intracorporus cavernosum, intradermal, intradiscal, intraductal, intraduodenal, intradural, intraepidermal, intraesophageal, intragastric, intragingival, intraileal, intralesional, intraluminal, intralymphatic, intramedullary, intrameningeal, intramuscular, intraocular, intraovarian, intrapericardial, intraperitoneal, intrapleural, intraprostatic, intrapulmonary, intrasinal, intraspinal, intrasynovial, intratendinous, intratesticular, intrathecal, intrathoracic, intratubular, intratumor, intratym panic, intrauterine, intravascular, intravenous, intravenous bolus, intravenous drip, intraventricular, intravesical, intravitreal, iontophoresis, irrigation, laryngeal, nasal, nasogastric, occlusive dressing technique, ophthalmic, oral, oropharyngeal, other, parenteral, percutaneous, periarticular, peridural, perineural, periodontal, rectal, respiratory (inhalation), retrobulbar, soft tissue, subarachnoid, subconjunctival, subcutaneous, sublingual, submucosal, topical, transdermal, transmucosal, transplacental, transtracheal, transtympanic, ureteral, urethral, and/or vaginal administration, and/or any combination of the above administration routes, which typically depends on the disease to be treated, subject being treated, and/or agent(s) being administered.

As used herein “cancer” can refer to one or more types of cancer including, but not limited to, acute lymphoblastic leukemia, acute myeloid leukemia, adrenocortical carcinoma, Kaposi Sarcoma, AIDS-related lymphoma, primary central nervous system (CNS) lymphoma, anal cancer, appendix cancer, astrocytomas, atypical teratoid/Rhabdoid tumors, basal cell carcinoma of the skin, bile duct cancer, bladder cancer, bone cancer (including but not limited to Ewing Sarcoma, osteosarcomas, and malignant fibrous histiocytoma), brain tumors, breast cancer, bronchial tumors, Burkitt lymphoma, carcinoid tumor, cardiac tumors, germ cell tumors, embryonal tumors, cervical cancer, cholangiocarcinoma, chordoma, chronic lymphocytic leukemia, chronic myelogenous leukemia, chronic myeloproliferative neoplasms, colorectal cancer, craniopharyngioma, cutaneous T-Cell lymphoma, ductal carcinoma in situ, endometrial cancer, ependymoma, esophageal cancer, esthesioneuroblastoma, extracranial germ cell tumor, extragonadal germ cell tumor, eye cancer (including, but not limited to, intraocular melanoma and retinoblastoma), fallopian tube cancer, gallbladder cancer, gastric cancer, gastrointestinal carcinoid tumor, gastrointestinal stromal tumors, central nervous system germ cell tumors, extracranial germ cell tumors, extragonadal germ cell tumors, ovarian germ cell tumors, testicular cancer, gestational trophoblastic disease, Hairy cell leukemia, head and neck cancers, hepatocellular (liver) cancer, Langerhans cell histiocytosis, Hodgkin lymphoma, hypopharyngeal cancer, islet cell tumors, pancreatic neuroendocrine tumors, kidney (renal cell) cancer, laryngeal cancer, leukemia, lip cancer, oral cancer, lung cancer (non-small cell and small cell), lymphoma, melanoma, Merkel cell carcinoma, mesothelioma, metastatic squamous cell neck cancer, midline tract carcinoma with and without NUT gene changes, multiple endocrine neoplasia syndromes, multiple myeloma, plasma cell neoplasms, mycosis fungoides, myelodyspastic syndromes, myelodysplastic/myeloproliferative neoplasms, chronic myelogenous leukemia, nasal cancer, sinus cancer, non-Hodgkin lymphoma, pancreatic cancer, paraganglioma, paranasal sinus cancer, parathyroid cancer, penile cancer, pharyngeal cancer, pheochromocytoma, pituitary cancer, peritoneal cancer, prostate cancer, rectal cancer, Rhabdomyosarcoma, salivary gland cancer, uterine sarcoma, Sezary syndrome, skin cancer, small intestine cancer, large intestine cancer (colon cancer), soft tissue sarcoma, T-cell lymphoma, throat cancer, oropharyngeal cancer, nasopharyngeal cancer, hypoharyngeal cancer, thymoma, thymic carcinoma, thyroid cancer, transitional cell cancer of the renal pelvis and ureter, urethral cancer, uterine cancer, vaginal cancer, cervical cancer, vascular tumors and cancer, vulvar cancer, and Wilms Tumor.

As used herein “cardiovascular diseases” can refer to a number of conditions: (1) Heart disease—heart and blood vessel disease (also called heart disease) includes numerous problems, many of which are related to a process called atherosclerosis; (2) Atherosclerosis is a condition that develops when a substance called plaque builds up in the walls of the arteries. This buildup narrows the arteries, making it harder for blood to flow through. If a blood clot forms, it can block the blood flow. This can cause a heart attack or stroke; (3) Heart attack—a heart attack occurs when the blood flow to a part of the heart is blocked by a blood clot. If this clot cuts off the blood flow completely, the part of the heart muscle supplied by that artery begins to die; (4) Stroke—an ischemic stroke (the most common type of stroke) occurs when a blood vessel that feeds the brain gets blocked, usually from a blood clot. When the blood supply to a part of the brain is cut off, some brain cells will begin to die. This can result in the loss of functions controlled by that part of the brain, such as walking or talking. A hemorrhagic stroke occurs when a blood vessel within the brain bursts. This is most often caused by uncontrolled hypertension (high blood pressure); (5) Heart failure—sometimes called congestive heart failure, means the heart is not pumping blood as well as it should; (6) Arrhythmia—refers to an abnormal heart rhythm. There are various types of arrhythmias. The heart can beat too slow, too fast or irregularly. Bradycardia, or a heart rate that's too slow, is when the heart rate is less than 60 beats per minute. Tachycardia, or a heart rate that's too fast, refers to a heart rate of more than 100 beats per minute.

As used herein “COPD” means chronic obstructive pulmonary disease, a chronic inflammatory lung disease that causes obstructed airflow from the lungs. Symptoms include breathing difficulty, cough, mucus (sputum) production and wheezing.

As used herein “kidney disease” also called chronic kidney disease or chronic kidney failure, involves a gradual loss of kidney function. The kidneys filter wastes and excess fluids from blood, which are then removed in urine. Advanced chronic kidney disease can cause dangerous levels of fluid, electrolytes and wastes to build up in a subject's body.

As used herein “sarcopenia” means a condition characterized by loss of skeletal muscle mass and function. Although it is primarily a disease of the elderly, its development may be associated with conditions that are not exclusively seen in older persons. Sarcopenia is a syndrome characterized by progressive and generalized loss of skeletal muscle mass and strength and it is strictly correlated with physical disability, poor quality of life and death. In conditions such as malignancy, rheumatoid arthritis and aging, hypogonadism, lean body mass is lost while fat mass may be preserved or even increased. The loss in muscle mass may be associated with increased body fat so that despite normal weight there is marked weakness, this is a condition called sarcopenic obesity.

As used herein, “chemotherapeutic agent” or “chemotherapeutic” refers to a therapeutic agent utilized to prevent or treat cancer, for purposes of example only, such as dexamethasone.

As used herein, “control” can refer to an alternative subject or sample used in an experiment for comparison purpose and included to minimize or distinguish the effect of variables other than an independent variable.

The term “optional” or “optionally” means that the subsequent described event, circumstance or substituent may or may not occur, and that the description includes instances where the event or circumstance occurs and instances where it does not.

As used herein, “dose,” “unit dose,” or “dosage” can refer to physically discrete units suitable for use in a subject, each unit containing a predetermined quantity of a pharmaceutical formulation thereof calculated to produce the desired response or responses in association with its administration.

The term “molecular weight”, as used herein, can generally refer to the mass or average mass of a material. If a polymer or oligomer, the molecular weight can refer to the relative average chain length or relative chain mass of the bulk polymer. In practice, the molecular weight of polymers and oligomers can be estimated or characterized in various ways including gel permeation chromatography (GPC) or capillary viscometry. GPC molecular weights are reported as the weight-average molecular weight (Mw) as opposed to the number-average molecular weight (M). Capillary viscometry provides estimates of molecular weight as the inherent viscosity determined from a dilute polymer solution using a particular set of concentration, temperature, and solvent conditions.

As used herein, “pharmaceutical formulation” refers to the combination of an active agent, compound, or ingredient with a pharmaceutically acceptable carrier or excipient, making the composition suitable for diagnostic, therapeutic, or preventive use in vitro, in vivo, or ex vivo.

As used herein, “pharmaceutically acceptable carrier or excipient” refers to a carrier or excipient that is useful in preparing a pharmaceutical formulation that is generally safe, non-toxic, and is neither biologically or otherwise undesirable, and includes a carrier or excipient that is acceptable for veterinary use as well as human pharmaceutical use. A “pharmaceutically acceptable carrier or excipient” as used in the specification and claims includes both one and more than one such carrier or excipient.

As used herein, “polymer” refers to molecules made up of monomers repeat units linked together. “Polymers” are understood to include, but are not limited to, homopolymers, copolymers, such as for example, block, graft, random and alternating copolymers, terpolymers, etc. and blends and modifications thereof. “A polymer” can be a three-dimensional network (e.g. the repeat units are linked together left and right, front and back, up and down), a two-dimensional network (e.g. the repeat units are linked together left, right, up, and down in a sheet form), or a one-dimensional network (e.g. the repeat units are linked left and right to form a chain). “Polymers” can be composed, natural monomers or synthetic monomers and combinations thereof. The polymers can be biologic (e.g. the monomers are biologically important (e.g. an amino acid), natural, or synthetic.

The terms “subject,” “individual,” and “patient” are used interchangeably herein to refer to a vertebrate, preferably a mammal, more preferably a human. Mammals include, but are not limited to, murines, simians, humans, farm animals, sport animals, and pets. Tissues, cells and their progeny of a biological entity obtained in vivo or cultured in vitro are also encompassed by the term “subject”.

As used herein, “substantially pure” can mean an object species is the predominant species present (i.e., on a molar basis it is more abundant than any other individual species in the composition), and preferably a substantially purified fraction is a composition wherein the object species comprises about 50 percent of all species present. Generally, a substantially pure composition will comprise more than about 80 percent of all species present in the composition, more preferably more than about 85%, 90%, 95%, and 99%. Most preferably, the object species is purified to essential homogeneity (contaminant species cannot be detected in the composition by conventional detection methods) wherein the composition consists essentially of a single species.

As used interchangeably herein, the terms “sufficient” and “effective,” can refer to an amount (e.g. mass, volume, dosage, concentration, and/or time period) needed to achieve one or more desired and/or stated result(s). For example, a therapeutically effective amount refers to an amount needed to achieve one or more therapeutic effects.

As used herein, “tangible medium of expression” refers to a medium that is physically tangible or accessible and is not a mere abstract thought or an unrecorded spoken word. “Tangible medium of expression” includes, but is not limited to, words on a cellulosic or plastic material, or data stored in a suitable computer readable memory form. The data can be stored on a unit device, such as a flash memory or CD-ROM or on a server that can be accessed by a user via, e.g. a web interface.

As used herein, “therapeutic” can refer to treating, healing, and/or ameliorating a disease, disorder, condition, or side effect, or to decreasing in the rate of advancement of a disease, disorder, condition, or side effect. A “therapeutically effective amount” can therefore refer to an amount of a compound that can yield a therapeutic effect.

As used herein, the terms “treating” and “treatment” can refer generally to obtaining a desired pharmacological and/or physiological effect. The effect can be, but does not necessarily have to be, prophylactic in terms of preventing or partially preventing a disease, symptom or condition thereof, such as cancer and/or indirect damage, such as Cachexia associated with chronic diseases and/or their therapies or sarcopenia associated with the aging process. This includes treating side-effects or impact from therapies directed to treating cachexia, sarcopenis, their related disorders, as well as effects of therapies aimed at preventing/remedying aging. The effect can be therapeutic in terms of a partial or complete cure of a disease, condition, symptom or adverse effect attributed to the disease, disorder, or condition. The term “treatment” as used herein covers any treatment of cancer and/or indirect damage, such as Cachexia associated with chronic diseases or sarcopenia associated with the aging process, and/or damage/injury associated with or resulting from their respective therapies, in a subject, particularly a human and/or companion animal, and can include any one or more of the following: (a) preventing the disease or damage from occurring in a subject which may be predisposed to the disease but has not yet been diagnosed as having it; (b) inhibiting the disease, i.e., arresting its development; and (c) relieving the disease, i.e., mitigating or ameliorating the disease and/or its symptoms or conditions. The term “treatment” as used herein can refer to both therapeutic treatment alone, prophylactic treatment alone, or both therapeutic and prophylactic treatment. Those in need of treatment (subjects in need thereof) can include those already with the disorder and/or those in which the disorder is to be prevented. As used herein, the term “treating”, can include inhibiting the disease, disorder or condition, e.g., impeding its progress; and relieving the disease, disorder, or condition, e.g., causing regression of the disease, disorder and/or condition. Treating the disease, disorder, or condition can include ameliorating at least one symptom of the particular disease, disorder, or condition, even if the underlying pathophysiology is not affected, such as treating the pain of a subject by administration of an analgesic agent even though such agent does not treat the cause of the pain.

As used herein, the terms “weight percent,” “wt %,” and “wt. %,” which can be used interchangeably, indicate the percent by weight of a given component based on the total weight of a composition of which it is a component, unless otherwise specified. That is, unless otherwise specified, all wt % values are based on the total weight of the composition. It should be understood that the sum of wt % values for all components in a disclosed composition or formulation are equal to 100. Alternatively, if the wt % value is based on the total weight of a subset of components in a composition, it should be understood that the sum of wt % values the specified components in the disclosed composition or formulation are equal to 100.

As used herein, “water-soluble”, generally means at least about 10 g of a substance is soluble in 1 L of water, i.e., at neutral pH, at 25° C.

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “one embodiment”, “an embodiment,” “an example embodiment,” means that a particular feature, structure or characteristic described in connection with the embodiment is included in at least one embodiment of the present disclosure. Thus, appearances of the phrases “in one embodiment,” “in an embodiment,” or “an example embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the disclosure. For example, in the appended claims, any of the claimed embodiments can be used in any combination.

All patents, patent applications, published applications, and publications, databases, websites and other published materials cited herein are hereby incorporated by reference to the same extent as though each individual publication, published patent document, or patent application was specifically and individually indicated as being incorporated by reference. KITS

Any of the compounds and/or formulations described herein can be presented as a combination kit. As used herein, the terms “combination kit” or “kit of parts” refers to the compounds, compositions, formulations, particles, cells and any additional components that are used to package, sell, market, deliver, and/or administer the combination of elements or a single element, such as the active ingredient, contained therein. Such additional components include, but are not limited to, packaging, syringes, blister packages, bottles, and the like. When one or more of the compounds, compositions, formulations, particles, cells, described herein or a combination thereof (e.g., agent(s)) contained in the kit are administered simultaneously, the combination kit can contain the active agent(s) in a single formulation, such as a pharmaceutical formulation, (e.g., a tablet, liquid preparation, dehydrated preparation, etc.) or in separate formulations. When the compounds, compositions, formulations, particles, and cells described herein or a combination thereof and/or kit components are not administered simultaneously, the combination kit can contain each agent or other component in separate pharmaceutical formulations. The separate kit components can be contained in a single package or in separate packages within the kit.

In some embodiments, the combination kit also includes instructions printed on or otherwise contained in a tangible medium of expression. The instructions can provide information regarding the content of the compounds and/or formulations, safety information regarding the content of the compounds and formulations (e.g., pharmaceutical formulations), information regarding the dosages, indications for use, and/or recommended treatment regimen(s) for the compound(s) and/or pharmaceutical formulations contained therein. In some embodiments, the instructions can provide directions and protocols for administering the compounds and/or formulations described herein to a subject in need thereof. In some embodiments, the instructions can provide one or more embodiments of the methods for administration of the pharmaceutical formulation thereof such as any of the methods described in greater detail elsewhere herein.

Muscle wasting (Cachexia or sarcopenia) is associated with reduced physical function, hospitalization and even death. Mitochondria are known to play a dominant role in driving muscle wasting disorders such as Cachexia and sarcopenia. However, currently there are no FDA approved therapies to prevent or treat muscle wasting disorders. Our discovery shows that quercetin can protect against muscle wasting via its ability to preserve mitochondrial homeostatic balance in a muscle wasting model.

Quercetin, a natural polyphenol found in various fruits and vegetables, has been recognized for its ability to increase mitochondrial biogenesis. Our data shows that quercetin can protect against cancer and chemotherapy-induced muscle mass loss through improving mitochondrial homeostatic balance in mice. Specifically, quercetin treated mice had a greater muscle mass and muscle cross sectional area in a cancer and chemotherapy model compared to vehicle treated mice.

Additionally, quercetin treated mice had a greater number and larger intermyofibrillar mitochondria with increased relative protein expression of mitochondrial complexes V (SEQ ID NO: 23), III (SEQ ID NO: 22), and II (SEQ ID NO: 21) as well as cytochrome c (SEQ ID NO: 20) expression.

Quercetin treated mice also had increased MFN1 (SEQ ID NO: 1) and reduced FIS1 (SEQ ID NO: 2) relative protein expression. These data provide strong support for the development of quercetin as an agent to prevent or treat Cachexia and sarcopenia through its actions on mitochondria.

A Cachexia diagnosis is associated with a doubling in hospital stay and increased healthcare cost. Indeed, cancer patients and most cachectic patients do not survive treatment. Unfortunately, complexity in treating Cachexia is amplified by both the underlying malignancy or chronic disease and the treatment therapy, which can independently promote cachexia. Quercetin, an organic polyphenolic flavonoid, has demonstrated anti-inflammatory and antioxidant properties with promise in protecting against cancer and chemotherapy-induced dysfunction; however, whether quercetin is efficacious in maintaining muscle mass in tumor-bearing animals receiving chemotherapy has not been investigated. C26 tumor-bearing mice were given 5-fluorouracil (5FU; 30 mg/kg of lean mass i.p.) concomitant with quercetin (Quer; 50 mg/kg of body weight via oral gavage) or vehicle. Both C26+5FU and C26+5FU+Quer had similar body weight loss; however, muscle mass and cross-sectional area was greater in C26+5FU+Quer compared to C26+5FU. Additionally, C26+5FU+Quer had a greater number and larger intermyofibrillar mitochondria with increased relative protein expression of mitochondrial complexes V (SEQ ID NO: 23), III (SEQ ID NO: 22), and II (SEQ ID NO: 21) as well as cytochrome c expression (SEQ ID NO: 20). C26+5FU+Quer also had increased MIFN1 (SEQ ID NO: 1) and reduced FIS1 (SEQ ID NO: 2) relative protein expression without apparent benefits to muscle inflammatory signaling. Our data suggest that quercetin protected against cancer and chemotherapy-induced muscle mass loss through improving mitochondrial homeostatic balance.

Cachexia, the unintentional loss of body weight with chronic disease, is prevalent in chronic obstructive pulmonary disease, heart failure, chronic kidney disease, HIV, and multiple cancers. See, Arthur, S. T., et al., One-year prevalence, comorbidities and cost of cachexia-related inpatient admissions in the USA. Drugs Context, 2014. 3: p. 212265 and von Haehling, S. and S. D. Anker, Prevalence, incidence and clinical impact of cachexia: facts and numbers-update 2014. J Cachexia Sarcopenia Muscle, 2014. 5(4): p. 261-3. Cachexia is associated with reduced physical function, decreased tolerance for treatment, and increased mortality. A Cachexia diagnosis is consistent with a doubling in duration of hospital stay and an additional $4,000.00/case compared to non-cachectic patients. See, Id. Indeed, ~₈₀% of cancer patients with a Cachexia diagnosis do not survive treatment and 20% of all cancer-related deaths can be attributed to cachexia. See, Arthur, S. T., et al., Cachexia among US cancer patients. J Med Econ, 2016. 19(9): p. 874-80. Additionally, the loss of lean mass with Cachexia impairs chemotherapy treatment tolerance and can exacerbate functional decrements leading to further functional dependencies and accrued health care cost. See, Vigano, A., et al., Survival prediction in terminal cancer patients: a systematic review of the medical literature. Palliat Med, 2000. 14(5): p. 363-74; Sealy, M. J., et al., Low muscle mass is associated with early termination of chemotherapy related to toxicity in patients with head and neck cancer. Clin Nutr, 2019; Dahele, M., et al., Objective physical activity and self-reported quality of lfe inpatients receiving palliative chemotherapy. J Pain Symptom Manage, 2007. 33(6): p. 676-85. Both the underlying cancer and its treatment can negatively impact muscle quality and mass, exacerbating cachexia's deleterious impact.

While our understanding of chemotherapy-induced Cachexia is still in its earliest mechanistic stages, it appears that the muscle mitochondria sit at a nexus of cancer and chemotherapy-induced skeletal muscle dysfunction. See, VanderVeen, B. N., D. K. Fix, and J. A. Carson, Disrupted Skeletal Muscle Mitochondrial Dynamics, Mitophagy, and Biogenesis during Cancer Cachexia: A Role for Inflammation. Oxid Med Cell Longev, 2017. 2017: p. 3292087; Barreto, R., et al., Cancer and Chemotherapy Contribute to Muscle Loss by Activating Common Signaling Pathways. Front Physiol, 2016. 7: p. 472; Barreto, R., et al., Chemotherapy-related cachexia is associated with mitochondrial depletion and the activation of ERK1/2 and p38 MAPKs. Oncotarget, 2016. 7(28): p. 43442-43460; Morton, A. B., et al., Mitochondrial accumulation of doxorubicin in cardiac and diaphragm muscle following exercise preconditioning. Mitochondrion, 2019. 45: p. 52-62. Preclinical models of cancer-Cachexia have demonstrated losses in mitochondrial content, biogenesis, dynamics, and function concomitant with reduced muscle function. See, VanderVeen and Vanderveen, B. N., et al., The Effect of Wheel Exercise on Functional Indices of Cachexia in Tumor-bearing Mice. Med Sci Sports Exerc, 2020. 52(11): p. 2320-2330; VanderVeen, B. N., et al., Skeletal muscle function during the progression of cancer cachexia in the male Apc(Min+) mouse. J Appl Physiol (1985), 2018. 124(3): p. 684-695; Counts, B. R., J. L. Halle, and J. A. Carson, Early—Onset Physical Inactivity and Metabolic Dysfunction in Tumor-bearing Mice Is Associated with Accelerated Cachexia. Med Sci Sports Exerc, 2022. 54(1): p. 77-88; and Brown, J. L., et al., Mitochondrial degeneration precedes the development of muscle atrophy in progression of cancer cachexia in tumour-bearing mice. J Cachexia Sarcopenia Muscle, 2017. More recently, cytotoxic chemotherapies, including doxorubicin and 5-fluorouracil (5FU) were demonstrated to impair mitochondrial function, and decrease content without the presence of the tumor environment. See Barreto and Morton. Recently, new models examining the combination of 5FU containing therapies with the colon-26 (C26) colorectal cancer model of cancer Cachexia has demonstrated indices of Cachexia even with stunted tumor growth. See, Wijler, L. A., et al., Specialized nutrition improves muscle function and physical activity without affecting chemotherapy efficacy in C26 tumour-bearing mice. J Cachexia Sarcopenia Muscle, 2021. 12(3): p. 796-810. See also, Murphy et al., Mechanisms of chemotherapy-induced muscle wasting in mice with cancer cachexia. JCSM Rapid Communications, 2022. 5(1): p. 102-116. Furthermore, they showed these deficits can be improved with a dietary intervention that did not impair the anticancer efficacy of chemotherapies. See, Id.

While new therapeutics continue are tested each year, unfortunately, there are currently no approved therapies for cancer cachexia. Although traditional nutritional support (i.e., hypercaloric diets) has not been effective in preventing cancer associated weight loss, specialized anti-inflammatory and antioxidant diets as well as nutraceuticals continue to be a safe alternative to pharmacological intervention with therapeutic promise. See, Id. Quercetin (3,4,5,7-pentahydroxylflavone) is an organic polyphenolic flavonoid commonly found in fruits and vegetables, such as grapes, apples, blueberries, and onions. See, Mattivi, F., et al., Metabolite profiling of grape: Flavonols and anthocyanins. J Agric Food Chem, 2006. 54(20): p. 7692-702; and Harnly, J. M., et al., Flavonoid content of U.S. fruits, vegetables, and nuts. J Agric Food Chem, 2006. 54(26): p. 9966-77. Pre-clinical studies have found it to demonstrate anti-inflammatory, antiviral, antioxidant, cardio-protective, anti-carcinogenic, and neuroprotective properties, among many others. See, Davis, J. M., E. A. Murphy, and M. D. Carmichael, Effects of the dietary flavonoid quercetin upon performance and health. Curr Sports Med Rep, 2009. 8(4): p. 206-13; Harwood, M., et al., A critical review of the data related to the safety of quercetin and lack of evidence of in vivo toxicity, including lack of genotoxic carcinogenic properties. Food Chem Toxicol, 2007. 45(11): p. 2179-205; Velazquez, K. T., et al., Quercetin supplementation attenuates the progression of cancer cachexia in ApcMin+ mice. J Nutr, 2014. 144(6): p. 868-75; Zern, T. L., et al., Grape polyphenols exert a cardioprotective effect in pre—and postmenopausal women by lowering plasma lipids and reducing oxidative stress. J Nutr, 2005. 135(8): p. 1911-7; Alexander, S. P., Flavonoids as antagonists at A1 adenosine receptors. Phytother Res, 2006. 20(11): p. 1009-12; Hou, D. D., et al., Anti-inflammatory effects of quercetin in a mouse model of MC903-induced atopic dermatitis. Int Immunopharmacol, 2019. 74: p. 105676; Ganesan, S., et al., Quercetin inhibits rhinovirus replication in vitro and in vivo. Antiviral Res, 2012. 94(3): p. 258-71; Bartekova, M., et al., Cardioprotective effects of quercetin against ischemia—reperfusion injury are age-dependent. Physiol Res, 2016. 65 Suppl 1: p. S101-7; Steiner, J., et al., Dose-dependent benefits of quercetin on tumorigenesis in the C3(1) SV40Tag transgenic mouse model of breast cancer. Cancer Biol Ther, 2014. 15(11): p. 1456-67; Murphy, E. A., et al., Quercetin's effects on intestinal polyp multiplicity and macrophage number in the Apc(Min+) mouse. Nutr Cancer, 2011. 63(3): p. 421-6; and Maciel, R. M., et al., Neuroprotective effects of quercetin on memory and anxiogenic-like behavior in diabetic rats: Role ofectonucleotidases and acetylcholinesterase activities. Biomed Pharmacother, 2016. 84: p. 559-568. These properties associated with quercetin have established it as a dietary agent or complementary medicine with potential to treat inflammatory diseases and cancer, as well as related conditions like cancer cachexia. See Velazquez, Murphy E. A., Davis, J. M., et al., Quercetin reduces susceptibility to influenza infection following stressful exercise. Am J Physiol Regul Integr Comp Physiol, 2008. 295(2): p. R505-9; Mahoney, S. E., et al., Dietary quercetin reduces chemotherapy-induced fatigue in mice. Integr Cancer Ther, 2014. 13(5): p. 417-24; and Levolger, S., et al., Quercetin supplementation attenuates muscle wasting in cancer-associated cachexia in mice. Nutrition and Healthy Aging, 2021. 6: p. 35-47.

We recently conducted a sub-chronic quercetin toxicity study in CD2F1 mice and found that quercetin had no deleterious effects or toxicities at various dosages. Cunningham, P., et al., Sub-chronic oral toxicity screening of quercetin in mice. BMC Complement Med Ther, 2022. 22(1): p. 279. The purpose of the current disclosure is to provide quercetin's efficacy in preventing Cachexia in C26 tumor-bearing mice treated with 5FU. We hypothesized that C26 tumor-bearing mice treated with 5FU would experience significant body weight and muscle mass loss with disrupted mitochondrial homeostasis; however, supplementing with 50 mg/kg quercetin as a complementary therapy with 5FU would stave off cachexia's progression. We found that C26 tumor-bearing mice given 5FU and quercetin had partially improved muscle mass, preserved myofibrillar cross section area (CSA), and protected muscle mitochondrial content compared to C26 tumor-bearing mice given 5FU alone.

### Materials and Methods

### Animals

Male (n=20) CD2F1 (CD2F1) hybrid mice were purchased from Charles River Laboratory (Raleigh, NC) at 10 weeks of age. Mice arrived at our facilities and were given AlN76 diet and allowed to acclimate to the new facilities and purified diet for 4 weeks. Mice were kept in ventilated cages (5 per cage), on a 12:12 h light/dark cycle, in a humidity and temperature control room (~22° C.) and given ad libitum access to water and food. Animal handling and experiments were performed to minimize pain and discomfort. At 14 weeks of age mice were separated into four groups following stratified random sampling for body weight and lean mass: 1) Control (n=5), 2) C26 (n=5), 3) C26+5FU (n=5), 4) C26+5FU+Quer (n=5). Control mice were non-tumor-bearing mice given PBS i.p. and propylene glycol p.o. as vehicle controls. Body weights were measured daily. Food weights per cage were measured daily and average food intake was calculated by the difference in food weight between days. After 10 days of C26 tumor growth, both 5FU and quercetin were given daily for 5 days until Day 15. Mice were euthanized 24 hours following the last 5FU injection (Day 16). Hind limb muscles and select organs were excised, weighed, and snap frozen in liquid nitrogen while mice were under sedation (2% Isoflurane, 2 L/min O₂). All procedures involving animals were reviewed and approved by the Institutional Animal Care and Usage Committee (IACUC) at the University of South Carolina in accordance with the American Association for Laboratory Animal Science.

### Cell Culture and Cell Implantation

C26 (colon 26 adenocarcinoma) cells were gifted from Dr. Andrea Bonetto's laboratory and grown in complete DMEM (10% fetal bovine serum, 1% penicillin/streptomycin). Cells were split at 70% confluency and passage 3 was used for cell implantation. On Day 0, CD2F1 mice were given a subcutaneous injection of 1×10⁶ C26 cells in the subscapular area as previously described. See, Bonetto, A., et al., The Colon-26 Carcinoma Tumor-bearing Mouse as a Model for the Study of Cancer Cachexia. J Vis Exp, 2016(117). Tumors were palpable at Day 8 in all C26 tumor-bearing mice.

### Chemotherapy Intervention

Fluorouracil (5FU; VWR; CAS #:51-21-8) was solubilized in warmed phosphate buffered saline (PBS) at 3.0 mg/mL, sterile filtered with a 0.2 μm filter and administered at the beginning of the light cycle (0700) via i.p. injection to mice with established tumor (day 11 after C26 implantation). We have previously demonstrated C57BL/6 mice are susceptible to 5FU-induced Cachexia at a dose of 40 mg/kg of lean mass (LM), see VanderVeen, B. N., et al., Obesity reduced survival with 5-fluorouracil and did not protect against chemotherapy—induced cachexia or immune cell cytotoxicity in mice. Cancer Biol Ther, 2022. 23(1): p. 1-15; and VanderVeen, B. N., et al., The Acute Effects of 5 Fluorouracil on Skeletal Muscle Resident and Infiltrating Immune Cells in Mice. Front Physiol, 2020. 11: p. 593468; however, CD2F1 mice were more sensitive to 5FU (data not shown) and therefore given 5FU at 30 mg/kg of LM.

### Quercetin Administration

Quercetin (Sigma-Aldrich; catalog #: Q4951) was made daily in propylene glycol at 4 mg/ml, concentration at which to not exceed maximum volume for oral gavage. Mice were given either quercetin at 50 mg/kg body weight (BW) or vehicle (propylene glycol) p.o. on Day 11 after C26 implantation at the end of the light cycle (1900).

### Dual Energy X-Ray Absorptiometry

Mice were subjected to Dual Energy X-ray Absorptiometry (DEXA; Lunar PIXI-mus) scan and image was then analyzed to assess lean body mass (LM). See VanderVeen, B. N.

### Muscle Histology

Skeletal muscle histology was assessed via hematoxylin and eosin (H&E) staining on tibialis anterior (TA) muscle cryosections. The TA muscle was dissected, snap-frozen in liquid nitrogen with Optimum Cutting Temperature (OCT) embedding medium placed on the most distal portion of the TA and stored in −80° C. Tissue was acclimated to the cryostat temperature (−24° C.) prior to collection of transverse sections (10 m) on Fisherbrand Superfrost Plus Microscope Slides. TA tissue slides were then stored at −80° C. until staining. Slides were fixed in ice-cold acetone and H&E staining was completed as previously described. See, VanderVeen, B. N., et al., Obesity reduced survival with 5-fluorouracil and did not protect against chemotherapy-induced cachexia or immune cell cytotoxicity in mice. Cancer Biol Ther, 2022. 23(1); and VanderVeen, B. N., et al., 5-Fluorouracil disrupts skeletal muscle immune cells and impairs skeletal muscle repair and remodeling. J Appl Physiol (1985), 2022. 133(4): p. 834-849. Images of H&E stains were taken at 20× and 40× magnification using a Keyence BZX800 microscope. Muscle cross sectional area (mCSA) was measured using ImageJ by measuring the circumference of 500-700 myofibers/mouse (n=3-5/group).

Myofiber Metabolic Phenotype

To assess skeletal muscle myofiber metabolic phenotype succinate dehydrogenase (SDH) activity was measured. SDH is an enzyme bound to the inner mitochondrial membrane and SDH activity is higher in type 1 fibers. See, Zogby, A. M., et al., Skeletal muscle fiber-type specific succinate dehydrogenase activity in cerebral palsy. Muscle Nerve, 2017. 55(1): p. 122-124. TA tissue cryosections were taken out of the −80° C. and air dried on the laboratory bench and then incubated in an SDH staining solution containing 0.5 mg/ml nitroblue tetrazolium and (Sigma, Cat #: N5514) and 50 mM sodium succinate (Sigma, Cat #: S2378) in PBS for 30 min at 37° C. See, Hardee, J. P., et al., Eccentric contraction-induced myofiber growth in tumor—bearing mice. J Appl Physiol (1985), 2016. 120(1): p. 29-37. Slides were then washed in distilled water 3×1 min before mounting with warmed glycerol mounting media and cover slipped. Images of SDH stains were taken at 4×, 20×, and 40× using a Keyence BZX800 microscope. SDH activity was analyzed by measuring the circumference and mean grey value of ~300 fibers from throughout the entire muscle. Images were inverted thus higher values corresponded with higher SDH activity.

### Electron Microscopy

Mitochondrial number and size were assessed in the soleus muscle using transmission electron microscopy (TEM). Skeletal muscle was prepared for TEM as previously described. See, VanderVeen, B. N., et al., 5-Fluorouracil disrupts skeletal muscle immune cells and impairs skeletal muscle repair and remodeling. J Appl Physiol (1985), 2022. 133(4): p. 834-849. The soleus was excised and initially fixed in 2.5% EM-grade glutaraldehyde followed by a secondary fixation in 1% osmium tetroxide and 1.5% K+ ferricyanide. After dehydration with increasing concentrations of ethanol, the samples were transferred to acetonitrile and embedded in Polybed 812. Ultrathin (~70 nm) sections were cut with a Leica Reichert Ultracut R ultramicrotome (Leica Microsystems GmbH, Wetzlar, Germany), collected on copper grids and stained with uranyl acetate and lead citrate. A JEOL 1400+ transmission electron microscope (JEOL USA, Peabody, MA) was used to view sections, and digital images were obtained with an Advanced Microscopy Techniques XR 81 Camera (Advanced Microscopy Techniques, Woburn, MA). Tissues were searched for intact and aligned sarcomeres by an investigator blinded to the experimental groups. For each mouse (n=3/group), ≥5 images were taken at 6000× across 3-5 myofibers within a single sagittal section. Mitochondrial number was taken by counting the number of discernible mitochondria within the field of view (F.O.V.) and averaged across the 5-7 images within a single mouse. Mitochondrial size was calculated by circling discernible mitochondria with ImageJ within the F.O.V. and averaged across the multiple images within a single mouse. Representative 1500× images were taken to further demonstrate distinguishable changes in mitochondrial number and size.

### Western Blotting

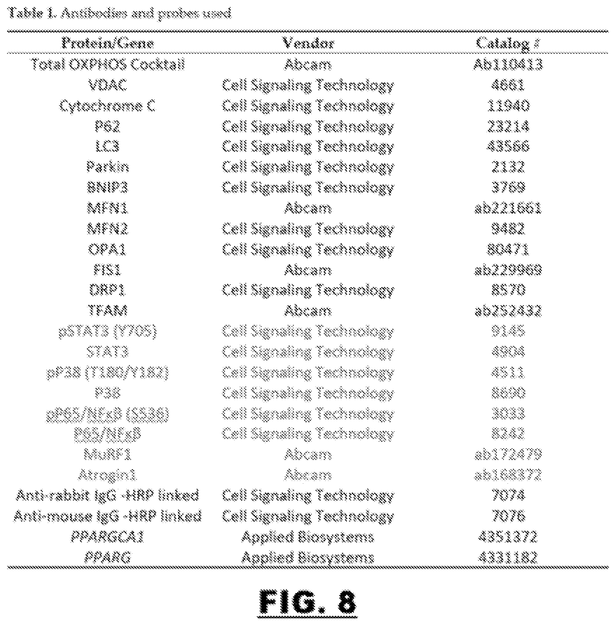

Protein isolation and western blotting was completed similar to what has been previously described. See, Sougiannis, A. T., et al., Impact of weight loss and partial weight regain on immune cell and inflammatory markers in adipose tissue in male mice. J Appl Physiol (1985), 2020. 129(4): p. 909-919. The rectus femoris (RF) was teased from the other quadriceps muscles, cut in half (proximal/distal), snap frozen in liquid nitrogen, and stored at −80° C. The distal RF was homogenized in Mueller Buffer (50 mM Hepes, 0.1% Triton-x, 4 mM EGTA, 10 mM EDTA, 15 mM Na4P207, 100 mM β-glycerophosphate, 25 mM NaF, and 5 mM NaVO₄, with protease inhibitor cocktail) using a bead homogenizer at 4° C. Samples were centrifuged and the supernatant was collected and diluted with a diluent buffer (50% Glycerol, 50 mM Na₄P₂O₇, 2.5 mM EGTA, and 1 mM β-mercaptoethanol with protease inhibitor cocktail). Protein concentrations were determined using the Bradford standard protein assay and protein integrity was confirmed with SDS-PAGE and amido black staining. Between 10-50 μg of muscle protein homogenate was separated via SDS-PAGE and transferred to a PVDF membrane. Membranes were stained with Ponceau Red to confirm even loading and transfer efficiency. Membranes were then washed with tris buffered saline with 0.1% Tween (TBST) before blocking for 1 h at room temp with 5% milk TBST. Membranes were then incubated in primary anti-body (Table 1, see FIG. 8) overnight at 4° C. under gentle agitation. Membranes were then washed 3×5 min and incubated with the appropriate (anti-mouse or rabbit) HRP-linked secondary in 5% milk TBST for 1 h at room temperature. After a final 3×5 min wash membranes were incubated in chemiluminescent HRP substrate before visualization with a Syngene G:box. Images were then scanned, and relative protein expression was determined with ImageJ. Investigator was blinded to sample loading scheme and images were cropped after analysis for representation. All samples (full Ns) were run together for each protein. Dotted lines indicate where gels were cropped.

Real time—polymerase chain reaction (RT-PCR)

RNA from the proximal portion of the RF was isolated as previously described. See, VanderVeen, B. N., et al., 5-Fluorouracil disrupts skeletal muscle immune cells and impairs skeletal muscle repair and remodeling. J Appl Physiol (1985), 2022. 133(4): p. 834-849. Briefly, RNA was extracted from the RF using the TRIzol/isopropanol/chloroform procedure (Life Technologies, Gibco-BRL, Carlsbad, CA). RNA sample quality and quantities were verified using a Nanodrop One Microvolume UV-Vis Spectrophotometer (Thermo Fisher Scientific, Waltham, MA). Samples with A260/A280 and A260/A230 values >1.8 were used for cDNA synthesis using High-capacity Reverse Transcriptase kit (Applied Biosystems, Foster City, CA). Quantitative RT-PCR analysis was carried out as per the manufacturer's instructions (Applied Biosystems) using Taq-Man Gene Expression Assays. Data were normalized to vehicle-treated controls and compared with five reference targets (B2M, TBP, HPRT, 18s, and H2AFV), which were evaluated for expression stability using the GeNorm algorithm.

### Plasma Interleukin-6 Analysis

Blood was collected retro orbitally prior to euthanasia while the mice were anesthetized. Whole blood was collected and put in an EDTA-coated tubes and store on ice prior to centrifugation (3,000 g at 4° C. for 10 min) for plasma collection. A commercially available IL-6 (SEQ ID NO: 9) enzyme-linked immunosorbent assay (ELISA) kit was obtained from BioLegend (San Diego, CA, USA). Briefly, a Costar clear 96-well plate (Corning, NY, USA) was coated with IL-6 (SEQ ID NO: 9) capture antibody and allowed to incubate overnight. The plate was then blocked with assay diluent buffer, and IL-6 (SEQ ID NO: 9) standards and plasma samples were added to the plate. Wells were then washed and detection antibody was incubated in each well for 1 h. The wells were again washed and then incubated with avidin horseradish peroxidase reagent. After several washes, 3,3′,5,5′-tetramethylbenzidine substrate was added, and the reaction was developed for 20 min. The reaction was stopped with sulfuric acid, and absorbance was measured.

### Statistical Analyses

Data were analyzed using Prism 8 statistical software (GraphPad Software, CA, USA). Unpaired t-test was used to determine differences between C26+5FU and C26+5FU+Quer. The healthy controls are shown as a reference point but were not included in statistical analysis. Additionally, the C26 alone group was not included in analysis, but is shown as a reference point for tumor growth without chemotherapy intervention. Data are presented as the mean±SEM and the level of significance was set up at p<0.05.

### Results

### Animal Characteristics

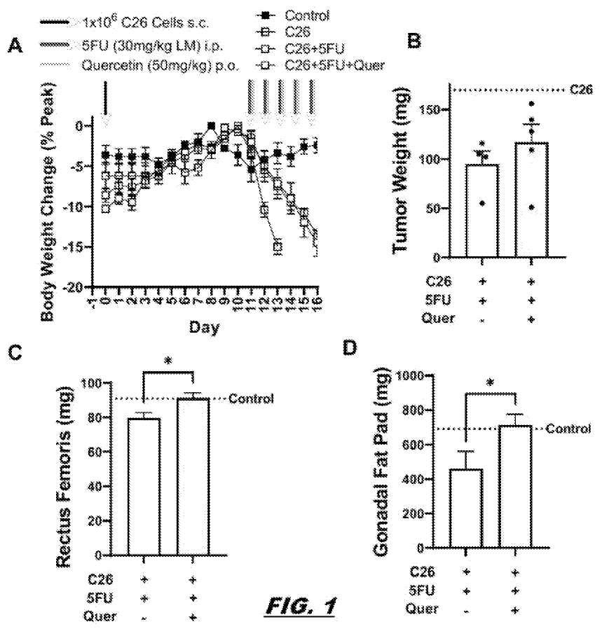

At 14 weeks of age, male CD2F1 mice were injected with 1×10⁶ C26 cells (Day 0) and body weights were tracked 16 days. At Day 11, 3 d after initial tumor palpation, mice were given daily i.p. injections of 5FU at 30 mg/kg of lean mass along with either propylene glycol or 50 mg/kg of quercetin (See FIG. 1 at A). C26 mice did not survive to Day 16, thus tumor and tissue weights were collected at Day 13. In mice given 5FU, acute administration of quercetin had no apparent impact on body weight loss (See FIG. 1 at A), nor did it impact 5FU's tumor suppressive capabilities (See FIG. 1 at B). Quercetin did, however, spare RF muscle weight (See FIG. 1 at C), and gonadal fat pad weight (See FIG. 1 at D). C26+5FU+Quer mice had 14.4% greater RF weight (p=0.02) compared to C26+5FU mice (See FIG. 1 at C). C26+5FU+Quer mice had 50.0% greater gonadal fat pad weight (p=0.04) compared to C26+5FU (See FIG. 1 at D). On average, C26+5FU mice consumed 1.05 g of food/d meanwhile C26+5FU+Quer ingested 0.94 g of food/d. Non-tumor bearing control mice consumed an average of 2.94 g of food/d.

FIG. 1 shows quercetin's impact on body and tissue weights. FIG. 1 shows at: A) Body weights shown as a percent of each mouse's peak body weight over the course of the study. Mice were injected with 1×10⁶ C26 cells subcutaneously (s.c.) at Day 0. Mice were then given 5FU at 30 mg/kg of lean mass (LM) via intraperitoneal (i.p.) injection starting at Day 11. Mice were also administered either quercetin (n=5) at 50 mg/kg body weight or propylene glycol (n=5) via oral gavage (per os; p.o.) starting at Day 11; at B) Tumor weights in milligrams (mg) taken at euthanasia. An additional cohort of mice given C26 without 5FU are shown as reference point (dotted line) for tumor growth without intervention; at C) Rectus femoris; and at D) gonadal fat pad weight in mg taken at euthanasia. n=5/group. Unpaired t-test between C26+5FU and C26+5FU+Quer. *p≤0.05.

### Quercetin Maintained Skeletal Muscle Cross Sectional Area

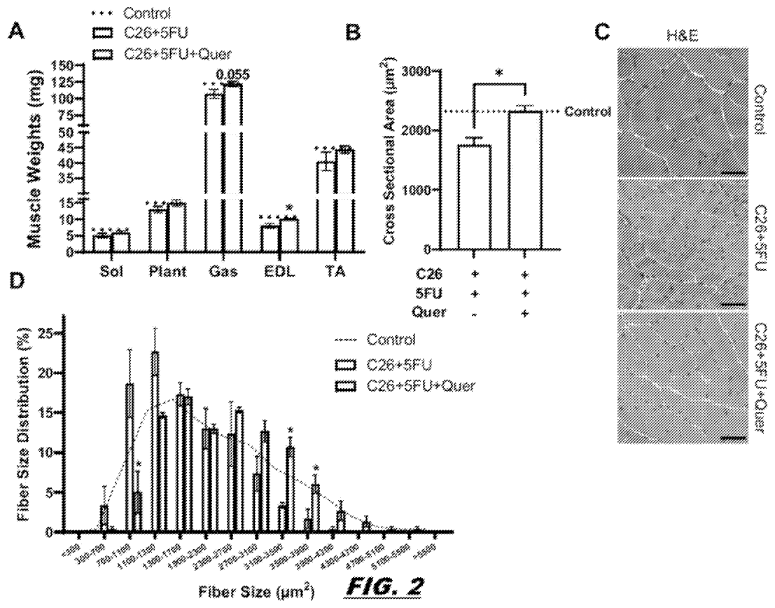

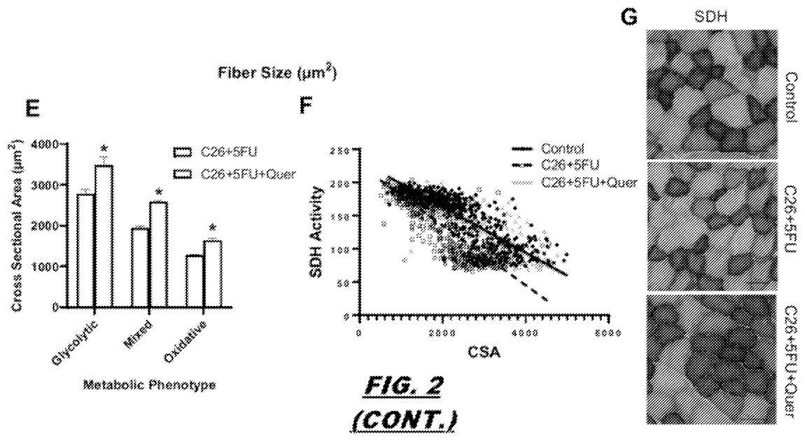

We then sought to further examine the anti-cachectic effects of quercetin. C26+5FU+Quer mice had 13.9% greater gastrocnemius weight (p=0.055) and 23.7% greater EDL weight (p=0.039) compared to C26+5FU mice (See FIG. 2 at A). While the other measured muscle weights did not achieve statistically significant differences (p=0.09-0.15), we sought to improve our sensitivity of detecting changes in muscle size by examining CSA. There was a 32.0% increase in TA mean CSA in C26+5FU+Quer compared to C26+5FU (See FIG. 2 at B, C). This can also be observed by a rightward shift in myofibrillar fiber size distribution in C26+5FU+Quer compared to C26+5FU (See FIG. 2 at D). We then sought to determine if there was a preferential sparing of glycolytic or oxidative fibers, and observed an increase in CSA across glycolytic, mixed, and oxidative fibers according to SDH activity (See FIG. 2 at E, G). Again, this can also be appreciated by examining the relationship between CSA and SDH activity demonstrating that C26+5FU+Quer had increased mCSA and SDH across the spectrum of fibers analyzed (See FIG. 2 at F).

FIG. 2 shows quercetin increased muscle cross sectional area and succinate dehydrogenase activity. FIG. 2 shows at: A) Hind limb muscle weights given in milligrams (mg)—soleus (Sol), plantaris (Plant), gastrocnemius (Gas), extensor digitorum longus (EDL), and tibialis anterior (TA); at B) Mean myofibrillar cross sectional area (CSA) in micrometers² (μm²) obtained from; at C) hematoxylin and eosin stained (H&E) TAs; at D) myofibrillar size distribution given in relative percent across increasing fiber sizes (μm²); at E) Mean CSA in μm² across glycolytic, mixed, or oxidative fiber types obtained from succinate dehydrogenase (SDH) stained TAs; at F) plotted linear relationship between single myofiber SDH activity and fiber size; and at G) Representative SDH images. Scale bar−50 μm. Unpaired t-test between C26+5FU and C26+5FU+Quer. n=3-5/group. *p≤0.05.

### Quercetin Maintained Skeletal Muscle Mitochondrial Size and Number

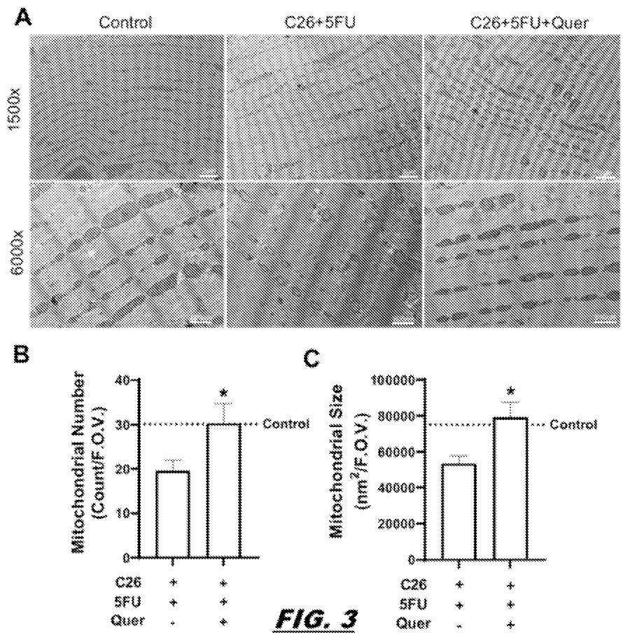

Quercetin has been hypothesized to protect against skeletal muscle wasting through improved oxidative metabolism and mitochondrial homeostasis. Therefore, we examined the impact of quercetin on mitochondrial size and number by TEM (See FIG. 3 at A). C26+5FU+Quer had 89.6% greater number of mitochondria (p=0.04; See FIG. 3 at B) and 45.3% greater mitochondrial size (p>0.01; See FIG. 3 at C).

FIG. 3 shows quercetin increased mitochondrial size and number. FIG. 3 shows at: A) Representative transmission electron microscopy images of the soleus muscle taken at 1500× and 6000×; at B) Average number of mitochondria per field of view (F.O.V.) from 6000× images; at C) Average size in nanometers² (nm²) of mitochondria per F.O.V. n=3/group. 1500× scale bar−2 μm. 6000× scale bar−600 nm. Unpaired t-test between C26+5FU and C26+5FU+Quer. n=3/group *p≤0.05.

### Quercetin Improved Mitochondrial Content-Associated Skeletal Muscle Proteins

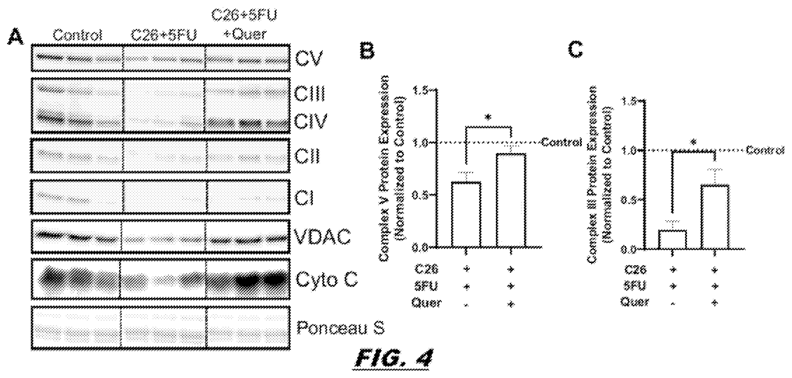

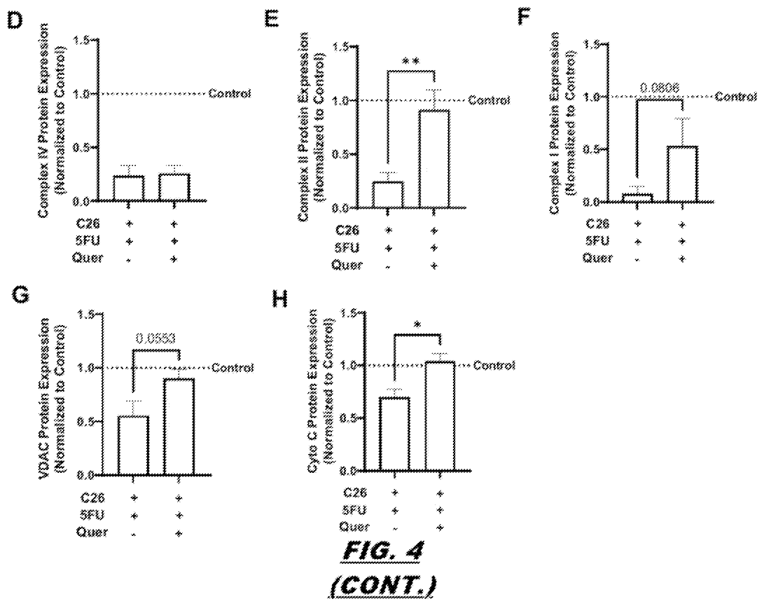

To further support the TEM results, we examined mitochondrial content proteins in whole muscle homogenates (See FIG. 4 at A). C26+5FU+Quer had increased relative expression of complex V (SEQ ID NO: 23) (43.9%; p=0.036; See FIG. 4 at B), III (SEQ ID NO: 22) (237%; p=0.024; See FIG. 4 at B), and II (SEQ ID NO: 21), (270%; p<0.01; See FIG. 4 at B) and a trending increase in complex I (SEQ ID NO: 24), (618%; p=0.08; See FIG. 4 at B) with no apparent changes to complex IV (SEQ ID NO: 25), (8.3%; p=0.44; See FIG. 4 at F) when compared to C26+5FU. Additionally, C26+5FU+Quer had increased relative expression of voltage dependent anion channel (VDAC (SEQ ID NO: 19)) by 62.8% (p=0.055; See FIG. 4 at G) and increased expression of cytochrome c (Cyto c) (SEQ ID NO: 20) by 48.9% (p=0.01; See FIG. 4 at H).

FIG. 4 shows quercetin increased mitochondrial content proteins. FIG. 4 show at: A) Representative western blots of mitochondrial complexes I-V (SEQ ID NOS: 21-25, voltage dependent anion channel (VDAC) (SEQ ID NO: 19), cytochrome c (Cyto C) (SEQ ID NO: 20), and loading control Ponceau S from the rectus femoris muscle. Vertical dotted line demonstrates where images were cropped for representation. All samples were run on the same gel/membrane. Quantified relative expression of, at B) Complex V (SEQ ID NO: 23); at C) Complex III (SEQ ID NO: 22); at D) Complex IV (SEQ ID NO: 25); at E) Complex II (SEQ ID NO: 21); at F) Complex I (SEQ ID NO: 24); at G) VDAC (SEQ ID NO: 19); and at H) Cyto C. Unpaired t-test between C26+5FU and C26+5FU+Quer. n=3-5/group. *p≤0.05.

### Quercetin Impacted Mitophagy-Associated Skeletal Muscle Proteins

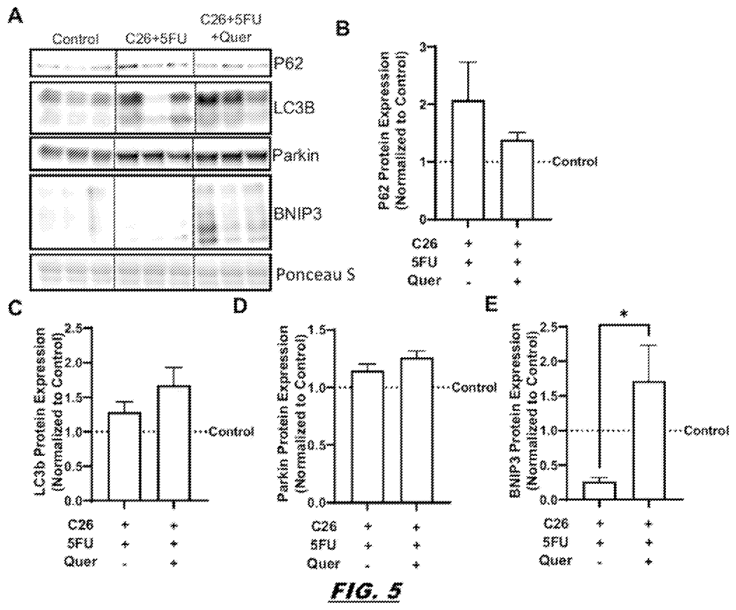

We then investigated the mechanisms by which quercetin improves muscle mitochondrial content. First, we examined proteins associated with autophagy and particularly mitochondrial associated autophagy (mitophagy; See FIG. 5 at A). There were no observed differences in general autophagy proteins, P62 (SEQ ID NO: 16) (−33.4%; p=0.20; See FIG. 5 at B) and LC3b (SEQ ID NO: 15) (−33.4%; p=0.2; See FIG. 5 at C). Examination of mitophagy specific proteins showed a trend to increase Parkin (SEQ ID NO: 17) expression by 10.2% (p=0.10; See FIG. 5 at D) and a significant 555% increase in BNIP3 (SEQ ID NO: 18) expression (p=0.024; See FIG. 5 at E) in C26+5FU+Quer compared to C26+5FU.

FIG. 5 shows quercetin has modest impacts of general autophagy and mitophagy proteins. FIG. 5 show at: A) Representative western blots of P62 (SEQ ID NO: 16), light chain 3 (LC3B (SEQ ID NO: 15)), Parkin (SEQ ID NO: 17), Bcl-2 interacting protein 3 (BNIP3 (SEQ ID NO: 18)), and loading control Ponceau S from the rectus femoris muscle. Vertical dotted line demonstrates where images were cropped for representation. All samples were run on the same gel/membrane. Quantified relative expression of B) P62 (SEQ ID NO: 16), C) LC3B (SEQ ID NO: 15), D) Parkin (SEQ ID NO: 17), and E) BNIP3 (SEQ ID NO: 18). Unpaired t-test between C26+5FU and C26+5FU+Quer. n=3-5/group. *p<0.05.

### Quercetin Differentially Impacted Mitochondrial Biogenesis, Fission, and Fusion

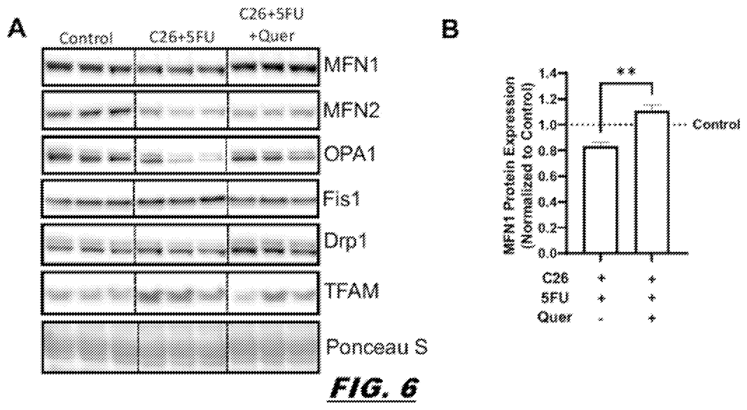

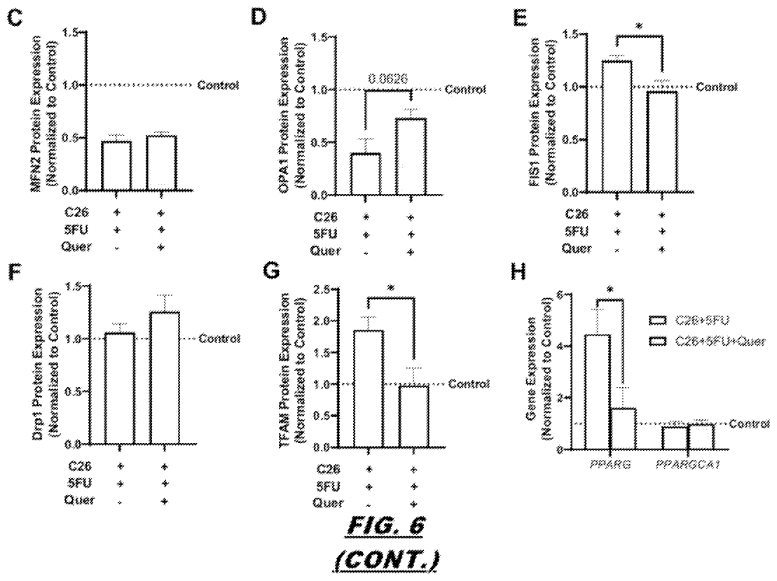

We further sought to investigate the mechanisms by which quercetin improves muscle mitochondrial content by examining mitochondrial dynamics proteins related to fission and fusion (See FIG. 6 at A). C26+5FU+Quer had increased relative expression of MFN1 (SEQ ID NO: 1) (33.3%; p=0.003; See FIG. 6 at B) without any apparent changes in MFN2 (SEQ ID NO: 3) (11.1%; p=0.25; See FIG. 6 at C). Additionally, there was a trend to increase OPA1 (SEQ ID NO: 4) (82.9%; p=0.06; See FIG. 6 at D). C26+5FU+Quer had reduced relative expression of FIS1 (SEQ ID NO: 2) (−23.4%; p=0.02; See FIG. 6 at E) compared to C26+5FU without any apparent changes to DRP1 (SEQ ID NO: 5) (18.9%; p=0.16; See FIG. 6 at F). C26+5FU+Quer had reduced relative expression of TFAM (SEQ ID NO: 6) (−47.5%; p=0.03; See FIG. 6 at G). C26+5FU+Quer also had reduced gene expression of PPARG (SEQ ID NO: 7) (−64.0%; p=0.04; See FIG. 6 at H) compared to C26+5FU without changes in PPARGCA1 (SEQ ID NO: 8) gene expression (10.3%; p=0.36; FIG. 6.

FIG. 6 shows quercetin normalizes mitochondrial dynamics proteins. FIG. 6 shows at: A) Representative western blots of mitofusion (MFN) 1 and 2, optic atrophy 1 (OPA1 (SEQ ID NO: 4)), mitochondrial fission 1 (FIS1 (SEQ ID NO: 2)), dynamin-related protein 1 (DRP1 (SEQ ID NO: 5)), mitochondrial transcription factor A (mtTFA/TFAM (SEQ ID NO: 6)), and loading control Ponceau S from the rectus femoris muscle. Vertical dotted line demonstrates where images were cropped for representation. All samples were run on the same gel/membrane. Quantified relative expression of B) MFN1 (SEQ ID NO: 1), C) MFN2 (SEQ ID NO: 3), D) OPA1 (SEQ ID NO: 4), E) FIS1 (SEQ ID NO: 2), F) DRP1 (SEQ ID NO: 5), G) TFAM (SEQ ID NO: 6), H) Relative gene expression of peroxisome proliferator activated receptor gamma (PPARγ/PPARG) (SEQ ID NO: 7) and PPARG co-activator 1 (PGC-la/PPARGCA1) (SEQ ID NO: 8). Unpaired t-test between C26+5FU and C26+5FU+Quer. n=3-5/group. *p<0.05.

### Quercetin has a Modest Impact on Skeletal Muscle Inflammatory Signaling

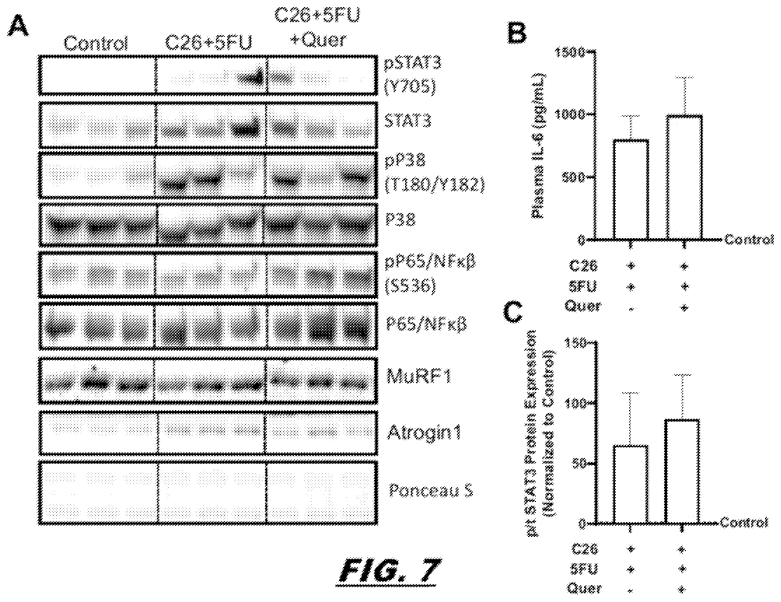

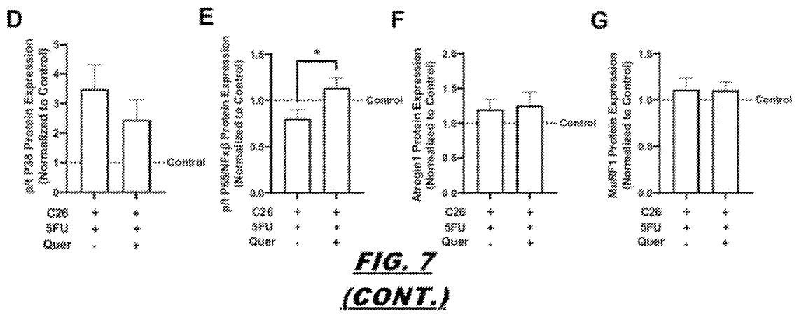

Quercetin has also been suggested to contain anti-inflammatory effects, which are thought to contribute to its anti-cachectic properties. Therefore, we examined the impact of quercetin on plasma IL-6 (SEQ ID NO: 9) (See FIG. 7 at A) and skeletal muscle inflammatory signaling proteins, STAT3 (SEQ ID NO: 10), P65/NFκβ (SEQ ID NO: 11), and P38/MAPK (SEQ ID NO: 12) (See FIG. 7 at B). It did not appear that quercetin decreased plasma IL-6 (SEQ ID NO: 9), phospho:total (p/t) STAT3 (SEQ ID NO: 10) (33.2%; p=0.36; See FIG. 7 at C) or P38 (−30.0%; p=0.19; See FIG. 7 at D); however, C26+5FU+Quer had increased p/tP65 (41.9%; p=0.03; See FIG. at E).

FIG. 7 shows quercetin has modest impact of circulating IL-6 (SEQ ID NO: 9) and muscle inflammatory signaling. FIG. 7 shows at A) representative western blots of phosphorylated (p; tyrosine 705) and total (t) signal transducer and activator of transcription 3 (STAT3 (SEQ ID NO: 10)), phosphorylated (serine 536) and total P65 (SEQ ID NO: 11), phosphorylated (threonine 180 and tyrosine 182) and total P38, muscle ring finger protein (MuRF) 1 (SEQ ID NO: 13), atrogin 1 (SEQ ID NO: 14), and loading control Ponceau S from the rectus femoris muscle. Vertical dotted line demonstrates where images were cropped for representation. All samples were run on the same gel/membrane; at B) Plasma interleukin 6 in picograms/milliliter (pg/mL). Quantified relative expression of C) p/tSTAT3 (SEQ ID NO: 10), D) p/tP38, E) p/tP65, F) MuRF1 (SEQ ID NO: 13), and G) Atrogin1 (SEQ ID NO: 14). Unpaired t-test between C26+5FU and C26+5FU+Quer. *p<0.05.

### DISCUSSION

Quercetin has been posed as a possible treatment of many inflammatory conditions, given its multi modal effects, see Davis, J. M., Harwood, M., and Velazquez, K. T., and its excellent safety profile. In the current disclosure, we sought to investigate quercetin's ability to protect against skeletal muscle mass loss in a novel model of cancer and chemotherapy-induced cachexia. We recently showed quercetin was safe at multiple doses, see Cunningham, P., and now show that quercetin partially preserved muscle mass and protected muscle mitochondrial content and quality control in the context of cancer and chemotherapy.

Skeletal muscle mitochondria appear central to cancer and chemotherapy-induced cachexia, see VanderVeen, B. N. and D. K. Fix, Barreto, R., Morton, A. B., Brown, J. L., Min, K., et al., Increased mitochondrial emission of reactive oxygen species and calpain activation are required for doxorubicin-induced cardiac and skeletal muscle myopathy. J Physiol, 2015. 593(8): p. 2017-36; Molinari, F., et al., The mitochondrial metabolic reprogramming agent trimetazidine as an ‘exercise mimetic’ in cachectic C26-bearing mice. J Cachexia Sarcopenia Muscle, 2017. 8(6): p. 954-973; Montalvo, R. N., et al., Doxorubicin-induced oxidative stress differentially regulates proteolytic signaling in cardiac and skeletal muscle. Am J Physiol Regul Integr Comp Physiol, 2020. 318(2): p. R227-R233; and Carson, J. A., J. P. Hardee, and B. N. VanderVeen, The emerging role of skeletal muscle oxidative metabolism as a biological target and cellular regulator of cancer-induced muscle wasting. Semin Cell Dev Biol, 2016. 54: p. 53-67. Quercetin has been shown to improve mitochondrial content and/or quality in several tissues. See, Davis, J. M., et al., Quercetin increases brain and muscle mitochondrial biogenesis and exercise tolerance. Am J Physiol Regul Integr Comp Physiol, 2009. 296(4): p. R1071-7; Chen, X., et al., Quercetin protects cardiomyocytes against doxorubicin-induced toxicity by suppressing oxidative stress and improving mitochondrial function via 14-3-3gamma. Toxicol Mech Methods, 2019. 29(5): p. 344-354; Cui, L., et al., Quercetin attenuates vascular calcification by inhibiting oxidative stress and mitochondrial fission. Vascul Pharmacol, 2017. 88: p. 21-29; Waseem, M., et al., Modulatory Role of Quercetin in Mitochondrial Dysfunction in Titanium Dioxide Nanoparticle-Induced Hepatotoxicity. ACS Omega, 2022. 7(4): p. 3192-3202; and Zymone, K., et al., Different Effects of Quercetin Glycosides and Quercetin on Kidney Mitochondrial Function-Uncoupling, Cytochrome C Reducing and Antioxidant Activity. Molecules, 2022. 27(19). Skeletal muscle mitochondrial content (demonstrated by mitochondria associated proteins Complex I-V (SEQ ID NOS: 21-25), VDAC (SEQ ID NO: 19), and Cyto C (SEQ ID NO: 20)) is primarily regulated by the balance of mitochondrial biogenesis (coordinated by PGC1-α and PPARγ) and autophagy/mitophagy (coordinated by LC3, P62 (SEQ ID NO: 16), Parkin (SEQ ID NO: 17), BNIP3 (SEQ ID NO: 18)), which involves the fusion of new or existing mitochondria to the mitochondrial network (coordinated by MFN1 (SEQ ID NO: 1), MNF2, and OPA1 (SEQ ID NO: 4)) or the fission of damaged or old mitochondria (coordinated by FIS1 (SEQ ID NO: 2) and DRP1 (SEQ ID NO: 5)). Mitochondrial function is often defined by the magnitude of ATP production in concert with H₂O₂ or free radical production. If ‘dysfunctional’, the mitochondria should be split and tagged by E3 ligase Parkin (SEQ ID NO: 17) or apoptotic BNIP3 (SEQ ID NO: 18) for removal by mitophagy. See, Romanello, V., et al., Mitochondrial fission and remodelling contributes to muscle atrophy. EMBO J, 2010. 29(10): p. 1774-85; Romanello, V. and M. Sandri, Mitochondrial biogenesis and fragmentation as regulators of protein degradation in striated muscles. J Mol Cell Cardiol, 2013. 55: p. 64-72; and Romanello, V. and M. Sandri, Mitochondrial Quality Control and Muscle Mass Maintenance. Front Physiol, 2015. 6: p. 422. Disruptions to these many processes have been demonstrated to disrupt skeletal muscle mass maintenance and function. See, Romanello, V. and M. Sandri, Mitochondrial Quality Control and Muscle Mass Maintenance. Front Physiol, 2015. 6: p. 422 and Leduc-Gaudet, J. P., et al., Mitochondrial Dynamics and Mitophagy in Skeletal Muscle Health and Aging. Int J Mol Sci, 2021. 22(15). In the current disclosure, we show that quercetin increased mitochondrial number and size assessed via TEM and increased overall content assessed via western blot. Additionally, we propose that this occurred through increased mitochondrial fusion and decreased fission.

In general, acute quercetin administration equilibrated the molecular signaling to protect against mitochondrial dysfunction. Our data show that quercetin increased BNIP3 (SEQ ID NO: 18) expression and decreased TFAM protein (SEQ ID NO: 6) and PPARG (SEQ ID NO: 7) gene expression when compared to vehicle treated C26+5FU mice. The increase of TFAM protein (SEQ ID NO: 6) and PPARG (SEQ ID NO: 7) gene expression observed in C26+5FU mice might be a compensation due to the loss of mitochondrial content, which is not apparent in the quercetin treated mice. Therefore, the compensatory upregulation of markers associated with mitochondrial biogenesis would not need to occur in quercetin treated mice. Similarly, the loss of BNIP3 (SEQ ID NO: 18) in C26+5FU mice could be due to a loss of mitochondrial content in which the mitochondrial specific BNIP3 (SEQ ID NO: 18) would be low in a whole muscle homogenate. BNIP3 (SEQ ID NO: 18) is thought to be located on the outer mitochondrial membrane, while Parkin (SEQ ID NO: 17) is located in the sarcoplasm and is recruited to the mitochondria, which helps explain why BNIP3 (SEQ ID NO: 18) was reduced while Parkin (SEQ ID NO: 17) was not changed. See, Romanello and Zhang, T. This is further supported by our results with general autophagy markers, P62 (SEQ ID NO: 16) and LC3.

Quercetin has been shown to improve muscle mass in cancer and rheumatoid arthritis preclinical models. See, Velazquez, K. T.; Levolger, S.; Manan, M., et al., Antiarthritic Potential of Comprehensively Standardized Extract of Alternanthera bettzickiana: In Vitro and In Vivo Studies. ACS Omega, 2020. 5(31): p. 19478-19496; Albrecht, C., M. C. Cittadini, and E. A. Soria, Pharmacological Activity of Quercetin and 5 Caffeoylquinic Acid Oral Intake in Male Balb c Mice with Lung Adenocarcinoma. Arch Med Res, 2020. 51(1): p. 8-12; Assi, M., et al., Free Radic Biol Med, 2016. 91: p. 204-14; Camargo, C. A., et al., Inhibition of tumor growth by quercetin with increase of survival and prevention of cachexia in Walker 256 tumor-bearing rats. Biochem Biophys Res Commun, 2011. 406(4): p. 638-42; and Francaux, M. and L. Deldicque, Using polyphenol derivatives to prevent muscle wasting. Curr Opin Clin Nutr Metab Care, 2018. 21(3): p. 159-163. Our group previously showed that 25 mg/kg of quercetin preserved muscle mass and strength in the ApcMin/+ model of intestinal cancer. See, Velazquez, K. T. Quercetin (35 mg/kg) also protected against muscle mass loss in the C26 model of cachexia. It was hypothesized that this sparing effect was due to a decrease in plasma IL-6 (SEQ ID NO: 9) which has been shown as a key driver of muscle mass loss and muscle mitochondrial loss in the ApcMin/+. See, VanderVeen, B. N., et al., The regulation of skeletal muscle fatigability and mitochondrial function by chronically elevated interleukin-6. Exp Physiol, 2019. 104(3): p. 385-397; White, J. P., et al., IL-6 regulation on skeletal muscle mitochondrial remodeling during cancer cachexia in the ApcMin+ mouse. Skelet Muscle, 2012. 2: p. 14; Baltgalvis, K. A., et al., Interleukin-6 and cachexia in ApcMin+ mice. Am J Physiol Regul Integr Comp Physiol, 2008. 294(2): p. R393-401. Differences observed in the inflammatory signaling might be due to the acute administration (5 oral gavages) of quercetin in the current disclosure. We show that quercetin did not reduce circulating IL-6 (SEQ ID NO: 9) or muscle inflammatory signaling which are both known regulators of skeletal muscle mass with cachexia, see, Narsale, A. A. and J. A. Carson, Role of interleukin-6 in cachexia: therapeutic implications. Curr Opin Support Palliat Care, 2014. 8(4): p. 321-7, and mitochondrial homeostasis balance can be modulated by inflammatory processes. See VanderVeen, B. N., D. K. Fix and VanderVeen, B. N. We then speculate that the improved mitochondrial control observed in the current disclosure is more likely due to the antioxidant properties of quercetin rather than its anti-inflammatory properties.

While the pursuit of novel pharmaceuticals continues in the effort to provide patients relief from cachexia's burden, complementary therapies including plant-based medicines and exercise provide safe and promising results on sparing muscle mass and function. See, Aquila, G., et al., Nutraceuticals and Exercise against Muscle Wasting during Cancer Cachexia. Cells, 2020. 9(12). In the current disclosure, we provide evidence in support of quercetin's anti-cachectic properties demonstrated by preserved muscle mass and CSA with improved mitochondrial content. While quercetin has been previously shown separately to improve cancer cachexia, see Velazquez, K. T., Levolger, S., Albrecht, C., and Camargo, C. A. and chemotherapy-induced fatigue, see Levolger S., et al., we are the first to demonstrate that quercetin was efficacious in a combined model of cancer and chemotherapy-induced skeletal muscle atrophy.

Various modifications and variations of the described methods, pharmaceutical compositions, and kits of the disclosure will be apparent to those skilled in the art without departing from the scope and spirit of the disclosure. Although the disclosure has been described in connection with specific embodiments, it will be understood that it is capable of further modifications and that the disclosure as claimed should not be unduly limited to such specific embodiments. Indeed, various modifications of the described modes for carrying out the disclosure that are obvious to those skilled in the art are intended to be within the scope of the disclosure. This application is intended to cover any variations, uses, or adaptations of the disclosure following, in general, the principles of the disclosure and including such departures from the present disclosure come within known customary practice within the art to which the disclosure pertains and may be applied to the essential features herein before set forth.

### SEQUENCE LISTING XML INCORPORATION STATEMENT

The herewith provided Sequence Listing XML is hereby incorporated by reference. The name of the XML file is USC1624.xml with a date of creation of 09/25/2023 and a size of 33 kilobytes.

<110> University of South Carolina
<120> TREATMENT REGIMENS AND METHODS OF USING QUERCETIN
TO PREVENT AND/OR TREAT MUSCLE WASTING DISORDERS
<130> 2033101.0000439
<140> Unknown
<141> ***
<150> U.S. Application No. 63/427,486
<151> 11/22/2022
<160> 1
<170> WIPO Sequence
<210> 1
<211> 741
<212> PRT
<213> Homo sapiens
<221> CDS
<222> 1...741
<400> 1
MetAlaGluProValSerProLeuLysHisPheValLeuAlaLysLysAlaIleThrAla
IlePheAspGlnLeuLeuGluPheValThrGluGlySerHisPheValGluAlaThrTyr
LysAsnProGluLeuAspArgIleAlaThrGluAspAspLeuValGluMetGlnGlyTyr
LysAspLysLeuSerIleIleGlyGluValLeuSerArgArgHisMetLysValAlaPhe
PheGlyArgThrSerSerGlyLysSerSerValIleAsnAlaMetLeuTrpAspLysVal
LeuProSerGlyIleGlyHisIleThrAsnCysPheLeuSerValGluGlyThrAspGly
AspLysAlaTyrLeuMetThrGluGlySerAspGluLysLysSerValLysThrValAsn
GlnLeuAlaHisAlaLeuHisMetAspLysAspLeuLysAlaGlyCysLeuValArgVal
PheTrpProLysAlaLysCysAlaLeuLeuArgAspAspLeuValLeuValAspSerPro
GlyThrAspValThrThrGluLeuAspSerTrpIleAspLysPheCysLeuAspAlaAsp
ValPheValLeuValAlaAsnSerGluSerThrLeuMetAsnThrGluLysHisPhePhe
HisLysValAsnGluArgLeuSerLysProAsnIlePheIleLeuAsnAsnArgTrpAsp
AlaSerAlaSerGluProGluTyrMetGluAspValArgArgGlnHisMetGluArgCys
LeuHisPheLeuValGluGluLeuLysValValAsnAlaLeuGluAlaGlnAsnArgIle
PhePheValSerAlaLysGluValLeuSerAlaArgLysGlnLysAlaGlnGlyMetPro
GluSerGlyValAlaLeuAlaGluGlyPheHisAlaArgLeuGlnGluPheGlnAsnPhe
GluGlnIlePheGluGluCysIleSerGlnSerAlaValLysThrLysPheGluGlnHis
ThrIleArgAlaLysGlnIleLeuAlaThrValLysAsnIleMetAspSerValAsnLeu
AlaAlaGluAspLysArgHisTyrSerValGluGluArgGluAspGlnIleAspArgLeu
AspPheIleArgAsnGlnMetAsnLeuLeuThrLeuAspValLysLysLysIleLysGlu
ValThrGluGluValAlaAsnLysValSerCysAlaMetThrAspGluIleCysArgLeu
SerValLeuValAspGluPheCysSerGluPheHisProAsnProAspValLeuLysIle
TyrLysSerGluLeuAsnLysHisIleGluAspGlyMetGlyArgAsnLeuAlaAspArg
CysThrAspGluValAsnAlaLeuValLeuGlnThrGlnGlnGluIleIleGluAsnLeu
LysProLeuLeuProAlaGlyIleGlnAspLysLeuHisThrLeuIleProCysLysLys
PheAspLeuSerTyrAsnLeuAsnTyrHisLysLeuCysSerAspPheGlnGluAspIle
ValPheArgPheSerLeuGlyTrpSerSerLeuValHisArgPheLeuGlyProArgAsn
AlaGlnArgValLeuLeuGlyLeuSerGluProIlePheGlnLeuProArgSerLeuAla
SerThrProThrAlaProThrThrProAlaThrProAspAsnAlaSerGlnGluGluLeu
MetIleThrLeuValThrGlyLeuAlaSerValThrSerArgThrSerMetGlyIleIle
IleValGlyGlyValIleTrpLysThrIleGlyTrpLysLeuLeuSerValSerLeuThr
MetTyrGlyAlaLeuTyrLeuTyrGluArgLeuSerTrpThrThrHisAlaLysGluArg
AlaPheLysGlnGlnPheValAsnTyrAlaThrGluLysLeuArgMetIleValSerSer
ThrSerAlaAsnCysSerHisGlnValLysGlnGlnIleAlaThrThrPheAlaArgLeu
CysGlnGlnValAspIleThrGlnLysGlnLeuGluGluGluIleAlaArgLeuProLys
GluIleAspGlnLeuGluLysIleGlnAsnAsnSerLysLeuLeuArgAsnLysAlaVal
GlnLeuGluAsnGluLeuGluAsnPheThrLysGlnPheLeuProSerSerAsnGluGlu
Ser
<210> 2
<211> 292
<212> PRTN
<213> Homo sapiens
<221> CDS
<222> 1-292
<400> 2
MetAsnProPheTrpSerMetSerThrSerSerValArgLysArgSerGluGlyGluGlu
LysThrLeuThrGlyAspValLysThrSerProProArgThrAlaProLysLysGlnLeu
ProSerIleProLysAsnAlaLeuProIleThrLysProThrSerProAlaProAlaAla
GlnSerThrAsnGlyThrHisAlaSerTyrGlyProPheTyrLeuGluTyrSerLeuLeu
AlaGluPheThrLeuValValLysGlnLysLeuProGlyValTyrValGlnProSerTyr
ArgSerAlaLeuMetTrpPheGlyValIlePheIleArgHisGlyLeuTyrGlnAspGly
ValPheLysPheThrValTyrIleProAspAsnTyrProAspGlyAspCysProArgLeu
ValPheAspIleProValPheHisProLeuValAspProThrSerGlyGluLeuAspVal
LysArgAlaPheAlaLysTrpArgArgAsnHisAsnHisIleTrpGlnValLeuMetTyr
AlaArgArgValPheTyrLysIleAspThrAlaSerProLeuAsnProGluAlaAlaVal
LeuTyrGluLysAspIleGlnLeuPheLysSerLysValValAspSerValLysValCys
ThrAlaArgLeuPheAspGlnProLysIleGluAspProTyrAlaIleSerPheSerPro
TrpAsnProSerValHisAspGluAlaArgGluLysMetLeuThrGlnLysLysProGlu
GluGlnHisAsnLysSerValHisValAlaGlyLeuSerTrpValLysProGlySerVal
GlnProPheSerLysGluGluLysThrValAlaThr
<210> 3
<211> 757
<212> PRTN
<213> Homo sapiens
<221> CDS
<222> 1-757
<400> 3
MetSerLeuLeuPheSerArgCysAsnSerIleValThrValLysLysAsnLysArgHis
MetAlaGluValAsnAlaSerProLeuLysHisPheValThrAlaLysLysLysIleAsn
GlyIlePheGluGlnLeuGlyAlaTyrIleGlnGluSerAlaThrPheLeuGluAspThr
TyrArgAsnAlaGluLeuAspProValThrThrGluGluGlnValLeuAspValLysGly
TyrLeuSerLysValArgGlyIleSerGluValLeuAlaArgArgHisMetLysValAla
PhePheGlyArgThrSerAsnGlyLysSerThrValIleAsnAlaMetLeuTrpAspLys
ValLeuProSerGlyIleGlyHisThrThrAsnCysPheLeuArgValGluGlyThrAsp
GlyHisGluAlaPheLeuLeuThrGluGlySerGluGluLysArgSerAlaLysThrVal
AsnGlnLeuAlaHisAlaLeuHisGlnAspLysGlnLeuHisAlaGlySerLeuValSer
ValMetTrpProAsnSerLysCysProLeuLeuLysAspAspLeuValLeuMetAspSer
ProGlyIleAspValThrThrGluLeuAspSerTrpIleAspLysPheCysLeuAspAla
AspValPheValLeuValAlaAsnSerGluSerThrLeuMetGlnThrGluLysHisPhe
PheHisLysValSerGluArgLeuSerArgProAsnIlePheIleLeuAsnAsnArgTrp
AspAlaSerAlaSerGluProGluTyrMetGluGluValArgArgGlnHisMetGluArg
CysThrSerPheLeuValAspGluLeuGlyValValAspArgSerGlnAlaGlyAspArg
IlePhePheValSerAlaLysGluValLeuAsnAlaArgIleGlnLysAlaGlnGlyMet
ProGluGlyGlyGlyAlaLeuAlaGluGlyPheGlnValArgMetPheGluPheGlnAsn
PheGluArgArgPheGluGluCysIleSerGlnSerAlaValLysThrLysPheGluGln
HisThrValArgAlaLysGlnIleAlaGluAlaValArgLeuIleMetAspSerLeuHis
MetAlaAlaArgGluGlnGlnValTyrCysGluGluMetArgGluGluArgGlnAspArg
LeuLysPheIleAspLysGlnLeuGluLeuLeuAlaGlnAspTyrLysLeuArgIleLys
GlnIleThrGluGluValGluArgGlnValSerThrAlaMetAlaGluGluIleArgArg
LeuSerValLeuValAspAspTyrGlnMetAspPheHisProSerProValValLeuLys
ValTyrLysAsnGluLeuHisArgHisIleGluGluGlyLeuGlyArgAsnMetSerAsp
ArgCysSerThrAlaIleThrAsnSerLeuGlnThrMetGlnGlnAspMetIleAspGly
LeuLysProLeuLeuProValSerValArgSerGlnIleAspMetLeuValProArgGln
CysPheSerLeuAsnTyrAspLeuAsnCysAspLysLeuCysAlaAspPheGlnGluAsp
IleGluPheHisPheSerLeuGlyTrpThrMetLeuValAsnArgPheLeuGlyProLys
AsnSerArgArgAlaLeuMetGlyTyrAsnAspGlnValGlnArgProIleProLeuThr
ProAlaAsnProSerMetProProLeuProGlnGlySerLeuThrGlnGluGluPheMet
ValSerMetValThrGlyLeuAlaSerLeuThrSerArgThrSerMetGlyIleLeuVal
ValGlyGlyValValTrpLysAlaValGlyTrpArgLeuIleAlaLeuSerPheGlyLeu
TyrGlyLeuLeuTyrValTyrGluArgLeuThrTrpThrThrLysAlaLysGluArgAla
PheLysArgGlnPheValGluHisAlaSerGluLysLeuGlnLeuValIleSerTyrThr
GlySerAsnCysSerHisGlnValGlnGlnGluLeuSerGlyThrPheAlaHisLeuCys
GlnGlnValAspValThrArgGluAsnLeuGluGlnGluIleAlaAlaMetAsnLysLys
IleGluValLeuAspSerLeuGlnSerLysAlaLysLeuLeuArgAsnLysAlaGlyTrp
LeuAspSerGluLeuAsnMetPheThrHisGlnTyrLeuGlnProSerArg
<210> 4
<211> 960
<212> PRTN
<213> Homo sapiens
<221> CDS
<222>
<400> 1-960
MetTrpArgLeuArgArgAlaAlaValAlaCysGluValCysGlnSerLeuValLysHis
SerSerGlyIleLysGlySerLeuProLeuGlnLysLeuHisLeuValSerArgSerIle
TyrHisSerHisHisProThrLeuLysLeuGlnArgProGlnLeuArgThrSerPheGln
GlnPheSerSerLeuThrAsnLeuProLeuArgLysLeuLysPheSerProIleLysTyr
GlyTyrGlnProArgArgAsnPheTrpProAlaArgLeuAlaThrArgLeuLeuLysLeu
ArgTyrLeuIleLeuGlySerAlaValGlyGlyGlyTyrThrAlaLysLysThrPheAsp
GlnTrpLysAspMetIleProAspLeuSerGluTyrLysTrpIleValProAspIleVal
TrpGluIleAspGluTyrIleAspPheGluLysIleArgLysAlaLeuProSerSerGlu
AspLeuValLysLeuAlaProAspPheAspLysIleValGluSerLeuSerLeuLeuLys
AspPhePheThrSerGlySerProGluGluThrAlaPheArgAlaThrAspArgGlySer
GluSerAspLysHisPheArgLysValSerAspLysGluLysIleAspGlnLeuGlnGlu
GluLeuLeuHisThrGlnLeuLysTyrGlnArgIleLeuGluArgLeuGluLysGluAsn
LysGluLeuArgLysLeuValLeuGlnLysAspAspLysGlyIleHisHisArgLysLeu
LysLysSerLeuIleAspMetTyrSerGluValLeuAspValLeuSerAspTyrAspAla
SerTyrAsnThrGlnAspHisLeuProArgValValValValGlyAspGlnSerAlaGly
LysThrSerValLeuGluMetIleAlaGlnAlaArgIlePheProArgGlySerGlyGlu
MetMetThrArgSerProValLysValThrLeuSerGluGlyProHisHisValAlaLeu
PheLysAspSerSerArgGluPheAspLeuThrLysGluGluAspLeuAlaAlaLeuArg
HisGluIleGluLeuArgMetArgLysAsnValLysGluGlyCysThrValSerProGlu
ThrIleSerLeuAsnValLysGlyProGlyLeuGlnArgMetValLeuValAspLeuPro
GlyValIleAsnThrValThrSerGlyMetAlaProAspThrLysGluThrIlePheSer
IleSerLysAlaTyrMetGlnAsnProAsnAlaIleIleLeuCysIleGlnAspGlySer
ValAspAlaGluArgSerIleValThrAspLeuValSerGlnMetAspProHisGlyArg
ArgThrIlePheValLeuThrLysValAspLeuAlaGluLysAsnValAlaSerProSer
ArgIleGlnGlnIleIleGluGlyLysLeuPheProMetLysAlaLeuGlyTyrPheAla
ValValThrGlyLysGlyAsnSerSerGluSerIleGluAlaIleArgGluTyrGluGlu
GluPhePheGlnAsnSerLysLeuLeuLysThrSerMetLeuLysAlaHisGlnValThr
ThrArgAsnLeuSerLeuAlaValSerAspCysPheTrpLysMetValArgGluSerVal
GluGlnGlnAlaAspSerPheLysAlaThrArgPheAsnLeuGluThrGluTrpLysAsn
AsnTyrProArgLeuArgGluLeuAspArgAsnGluLeuPheGluLysAlaLysAsnGlu
IleLeuAspGluValIleSerLeuSerGlnValThrProLysHisTrpGluGluIleLeu
GlnGlnSerLeuTrpGluArgValSerThrHisValIleGluAsnIleTyrLeuProAla
AlaGlnThrMetAsnSerGlyThrPheAsnThrThrValAspIleLysLeuLysGlnTrp
ThrAspLysGlnLeuProAsnLysAlaValGluValAlaTrpGluThrLeuGlnGluGlu
PheSerArgPheMetThrGluProLysGlyLysGluHisAspAspIlePheAspLysLeu
LysGluAlaValLysGluGluSerIleLysArgHisLysTrpAsnAspPheAlaGluAsp
SerLeuArgValIleGlnHisAsnAlaLeuGluAspArgSerIleSerAspLysGlnGln
TrpAspAlaAlaIleTyrPheMetGluGluAlaLeuGlnAlaArgLeuLysAspThrGlu
AsnAlaIleGluAsnMetValGlyProAspTrpLysLysArgTrpLeuTyrTrpLysAsn
ArgThrGlnGluGlnCysValHisAsnGluThrLysAsnGluLeuGluLysMetLeuLys
CysAsnGluGluHisProAlaTyrLeuAlaSerAspGluIleThrThrValArgLysAsn
LeuGluSerArgGlyValGluValAspProSerLeuIleLysAspThrTrpHisGlnVal
TyrArgArgHisPheLeuLysThrAlaLeuAsnHisCysAsnLeuCysArgArgGlyPhe
TyrTyrTyrGlnArgHisPheValAspSerGluLeuGluCysAsnAspValValLeuPhe
TrpArgIleGlnArgMetLeuAlaIleThrAlaAsnThrLeuArgGlnGlnLeuThrAsn
ThrGluValArgArgLeuGluLysAsnValLysGluValLeuGluAspPheAlaGluAsp
GlyGluLysLysIleLysLeuLeuThrGlyLysArgValGlnLeuAlaGluAspLeuLys
LysValArgGluIleGlnGluLysLeuAspAlaPheIleGluAlaLeuHisGlnGluLys
<210> 5
<211> 198
<212> PRTN
<213> Homo sapiens
<221> CDS
<222> 1-198
<400> 5
MetAlaAlaAspIleSerGluSerSerGlyAlaAspCysLysGlyAspProArgAsnSer
AlaLysLeuAspAlaAspTyrProLeuArgValLeuTyrCysGlyValCysSerLeuPro
ThrGluTyrCysGluTyrMetProAspValAlaLysCysArgGlnTrpLeuGluLysAsn
PheProAsnGluPheAlaLysLeuThrValGluAsnSerProLysGlnGluAlaGlyIle
SerGluGlyGlnGlyThrAlaGlyGluGluGluGluLysLysLysGlnLysArgGlyGly
ArgGlyGlnIleLysGlnLysLysLysThrValProGlnLysValThrIleAlaLysIle
ProArgAlaLysLysLysTyrValThrArgValCysGlyLeuAlaThrPheGluIleAsp
LeuLysGluAlaGlnArgPhePheAlaGlnLysPheSerCysGlyAlaSerValThrGly
GluAspGluIleIleIleGlnGlyAspPheThrAspAspIleIleAspValIleGlnGlu
LysTrpProGluValAspAspAspSerIleGluAspLeuGlyGluValLysLys
<210> 6
<211> 246
<212> PRTN
<213> Homo sapiens
<221> CDS
<222> 1-246
<400> 6
MetAlaPheLeuArgSerMetTrpGlyValLeuSerAlaLeuGlyArgSerGlyAlaGlu
LeuCysThrGlyCysGlySerArgLeuArgSerProPheSerPheValTyrLeuProArg
TrpPheSerSerValLeuAlaSerCysProLysLysProValSerSerTyrLeuArgPhe
SerLysGluGlnLeuProIlePheLysAlaGlnAsnProAspAlaLysThrThrGluLeu
IleArgArgIleAlaGlnArgTrpArgGluLeuProAspSerLysLysLysIleTyrGln
AspAlaTyrArgAlaGluTrpGlnValTyrLysGluGluIleSerArgPheLysGluGln
LeuThrProSerGlnIleMetSerLeuGluLysGluIleMetAspLysHisLeuLysArg
LysAlaMetThrLysLysLysGluLeuThrLeuLeuGlyLysProLysArgProArgSer
AlaTyrAsnValTyrValAlaGluArgPheGlnGluAlaLysGlyAspSerProGlnGlu
LysLeuLysThrValLysGluAsnTrpLysAsnLeuSerAspSerGluLysGluLeuTyr
IleGlnHisAlaLysGluAspGluThrArgTyrHisAsnGluMetLysSerTrpGluGlu
GlnMetIleGluValGlyArgLysAspLeuLeuArgArgThrIleLysLysGlnArgLys
TyrGlyAlaGluGluCys
<210> 7
<211> 505
<212> PRTN
<213> Homo sapiens
<221> CDS
<222> 1-505
<400> 7
MetGlyGluThrLeuGlyAspSerProIleAspProGluSerAspSerPheThrAspThr
LeuSerAlaAsnIleSerGlnGluMetThrMetValAspThrGluMetProPheTrpPro
ThrAsnPheGlyIleSerSerValAspLeuSerValMetGluAspHisSerHisSerPhe
AspIleLysProPheThrThrValAspPheSerSerIleSerThrProHisTyrGluAsp
IleProPheThrArgThrAspProValValAlaAspTyrLysTyrAspLeuLysLeuGln
GluTyrGlnSerAlaIleLysValGluProAlaSerProProTyrTyrSerGluLysThr
GlnLeuTyrAsnLysProHisGluGluProSerAsnSerLeuMetAlaIleGluCysArg
ValCysGlyAspLysAlaSerGlyPheHisTyrGlyValHisAlaCysGluGlyCysLys
GlyPhePheArgArgThrIleArgLeuLysLeuIleTyrAspArgCysAspLeuAsnCys
ArgIleHisLysLysSerArgAsnLysCysGlnTyrCysArgPheGlnLysCysLeuAla
ValGlyMetSerHisAsnAlaIleArgPheGlyArgMetProGlnAlaGluLysGluLys
LeuLeuAlaGluIleSerSerAspIleAspGlnLeuAsnProGluSerAlaAspLeuArg
AlaLeuAlaLysHisLeuTyrAspSerTyrIleLysSerPheProLeuThrLysAlaLys
AlaArgAlaIleLeuThrGlyLysThrThrAspLysSerProPheValIleTyrAspMet
AsnSerLeuMetMetGlyGluAspLysIleLysPheLysHisIleThrProLeuGlnGlu
GlnSerLysGluValAlaIleArgIlePheGlnGlyCysGlnPheArgSerValGluAla
ValGlnGluIleThrGluTyrAlaLysSerIleProGlyPheValAsnLeuAspLeuAsn
AspGlnValThrLeuLeuLysTyrGlyValHisGluIleIleTyrThrMetLeuAlaSer
LeuMetAsnLysAspGlyValLeuIleSerGluGlyGlnGlyPheMetThrArgGluPhe
LeuLysSerLeuArgLysProPheGlyAspPheMetGluProLysPheGluPheAlaVal
LysPheAsnAlaLeuGluLeuAspAspSerAspLeuAlaIlePheIleAlaValIleIle
LeuSerGlyAspArgProGlyLeuLeuAsnValLysProIleGluAspIleGlnAspAsn
LeuLeuGlnAlaLeuGluLeuGlnLeuLysLeuAsnHisProGluSerSerGlnLeuPhe
AlaLysLeuLeuGlnLysMetThrAspLeuArgGlnIleValThrGluHisValGlnLeu
LeuGlnValIleLysLysThrGluThrAspMetSerLeuHisProLeuLeuGlnGluIle
TyrLysAspLeuTyr
<210> 8
<211> 798
<212> PRTN
<213> Homo sapiens
<221> CDS
<222> 1-798
<400> 8
MetAlaTrpAspMetCysAsnGlnAspSerGluSerValTrpSerAspIleGluCysAla
AlaLeuValGlyGluAspGlnProLeuCysProAspLeuProGluLeuAspLeuSerGlu
LeuAspValAsnAspLeuAspThrAspSerPheLeuGlyGlyLeuLysTrpCysSerAsp
GlnSerGluIleIleSerAsnGlnTyrAsnAsnGluProSerAsnIlePheGluLysIle
AspGluGluAsnGluAlaAsnLeuLeuAlaValLeuThrGluThrLeuAspSerLeuPro
ValAspGluAspGlyLeuProSerPheAspAlaLeuThrAspGlyAspValThrThrAsp
AsnGluAlaSerProSerSerMetProAspGlyThrProProProGlnGluAlaGluGlu
ProSerLeuLeuLysLysLeuLeuLeuAlaProAlaAsnThrGlnLeuSerTyrAsnGlu
CysSerGlyLeuSerThrGlnAsnHisAlaAsnHisAsnHisArgIleArgThrAsnPro
AlaIleValLysThrGluAsnSerTrpSerAsnLysAlaLysSerIleCysGlnGlnGln
LysProGlnArgArgProCysSerGluLeuLeuLysTyrLeuThrThrAsnAspAspPro
ProHisThrLysProThrGluAsnArgAsnSerSerArgAspLysCysThrSerLysLys
LysSerHisThrGlnSerGlnSerGlnHisLeuGlnAlaLysProThrThrLeuSerLeu
ProLeuThrProGluSerProAsnAspProLysGlySerProPheGluAsnLysThrIle
GluArgThrLeuSerValGluLeuSerGlyThrAlaGlyLeuThrProProThrThrPro
ProHisLysAlaAsnGlnAspAsnProPheArgAlaSerProLysLeuLysSerSerCys
LysThrValValProProProSerLysLysProArgTyrSerGluSerSerGlyThrGln
GlyAsnAsnSerThrLysLysGlyProGluGlnSerGluLeuTyrAlaGlnLeuSerLys
SerSerValLeuThrGlyGlyHisGluGluArgLysThrLysArgProSerLeuArgLeu
PheGlyAspHisAspTyrCysGlnSerIleAsnSerLysThrGluIleLeuIleAsnIle
SerGlnGluLeuGlnAspSerArgGlnLeuGluAsnLysAspValSerSerAspTrpGln
GlyGlnIleCysSerSerThrAspSerAspGlnCysTyrLeuArgGluThrLeuGluAla
SerLysGlnValSerProCysSerThrArgLysGlnLeuGlnAspGlnGluIleArgAla
GluLeuAsnLysHisPheGlyHisProSerGlnAlaValPheAspAspGluAlaAspLys
ThrGlyGluLeuArgAspSerAspPheSerAsnGluGlnPheSerLysLeuProMetPhe
IleAsnSerGlyLeuAlaMetAspGlyLeuPheAspAspSerGluAspGluSerAspLys
LeuSerTyrProTrpAspGlyThrGlnSerTyrSerLeuPheAsnValSerProSerCys
SerSerPheAsnSerProCysArgAspSerValSerProProLysSerLeuPheSerGln
ArgProGlnArgMetArgSerArgSerArgSerPheSerArgHisArgSerCysSerArg
SerProTyrSerArgSerArgSerArgSerProGlySerArgSerSerSerArgSerCys
TyrTyrTyrGluSerSerHisTyrArgHisArgThrHisArgAsnSerProLeuTyrVal
ArgSerArgSerArgSerProTyrSerArgArgProArgTyrAspSerTyrGluGluTyr
GlnHisGluArgLeuLysArgGluGluTyrArgArgGluTyrGluLysArgGluSerGlu
ArgAlaLysGlnArgGluArgGlnArgGlnLysAlaIleGluGluArgArgValIleTyr
ValGlyLysIleArgProAspThrThrArgThrGluLeuArgAspArgPheGluValPhe
GlyGluIleGluGluCysThrValAsnLeuArgAspAspGlyAspSerTyrGlyPheIle
ThrTyrArgTyrThrCysAspAlaPheAlaAlaLeuGluAsnGlyTyrThrLeuArgArg
SerAsnGluThrAspPheGluLeuTyrPheCysGlyArgLysGlnPhePheLysSerAsn
TyrAlaAspLeuAspSerAsnSerAspAspPheAspProAlaSerThrLysSerLysTyr
AspSerLeuAspPheAspSerLeuLeuLysGluAlaGlnArgSerLeuArgArg
<210> 9
<211> 212
<212> PRTN
<213> Homo sapiens
<221> CDS
<222> 1-212
<400> 9
MetAsnSerPheSerThrSerAlaPheGlyProValAlaPheSerLeuGlyLeuLeuLeu
ValLeuProAlaAlaPheProAlaProValProProGlyGluAspSerLysAspValAla
AlaProHisArgGlnProLeuThrSerSerGluArgIleAspLysGlnIleArgTyrIle
LeuAspGlyIleSerAlaLeuArgLysGluThrCysAsnLysSerAsnMetCysGluSer
SerLysGluAlaLeuAlaGluAsnAsnLeuAsnLeuProLysMetAlaGluLysAspGly
CysPheGlnSerGlyPheAsnGluGluThrCysLeuValLysIleIleThrGlyLeuLeu
GluPheGluValTyrLeuGluTyrLeuGlnAsnArgPheGluSerSerGluGluGlnAla
ArgAlaValGlnMetSerThrLysValLeuIleGlnPheLeuGlnLysLysAlaLysAsn
LeuAspAlaIleThrThrProAspProThrThrAsnAlaSerLeuLeuThrLysLeuGln
AlaGlnAsnGlnTrpLeuGlnAspMetThrThrHisLeuIleLeuArgSerPheLysGlu
PheLeuGlnSerSerLeuArgAlaLeuArgGlnMet
<210> 10
<211> 770
<212> PRTN
<213> Homo sapiens
<221> CDS
<222> 1-770
<400> 10
MetAlaGlnTrpAsnGlnLeuGlnGlnLeuAspThrArgTyrLeuGluGlnLeuHisGln
LeuTyrSerAspSerPheProMetGluLeuArgGlnPheLeuAlaProTrpIleGluSer
GlnAspTrpAlaTyrAlaAlaSerLysGluSerHisAlaThrLeuValPheHisAsnLeu
LeuGlyGluIleAspGlnGlnTyrSerArgPheLeuGlnGluSerAsnValLeuTyrGln
HisAsnLeuArgArgIleLysGlnPheLeuGlnSerArgTyrLeuGluLysProMetGlu
IleAlaArgIleValAlaArgCysLeuTrpGluGluSerArgLeuLeuGlnThrAlaAla
ThrAlaAlaGlnGlnGlyGlyGlnAlaAsnHisProThrAlaAlaValValThrGluLys
GlnGlnMetLeuGluGlnHisLeuGlnAspValArgLysArgValGlnAspLeuGluGln
LysMetLysValValGluAsnLeuGlnAspAspPheAspPheAsnTyrLysThrLeuLys
SerGlnGlyAspMetGlnAspLeuAsnGlyAsnAsnGlnSerValThrArgGlnLysMet
GlnGlnLeuGluGlnMetLeuThrAlaLeuAspGlnMetArgArgSerIleValSerGlu
LeuAlaGlyLeuLeuSerAlaMetGluTyrValGlnLysThrLeuThrAspGluGluLeu
AlaAspTrpLysArgArgGinGinIleAlaCysIleGlyGlyProProAsnIleCysLeu
AspArgLeuGluAsnTrpIleThrSerLeuAlaGluSerGlnLeuGlnThrArgGlnGln
IleLysLysLeuGluGluLeuGlnGlnLysValSerTyrLysGlyAspProIleValGln
HisArgProMetLeuGluGluArgIleValGluLeuPheArgAsnLeuMetLysSerAla
PheValValGluArgGlnProCysMetProMetHisProAspArgProLeuValIleLys
ThrGlyValGlnPheThrThrLysValArgLeuLeuValLysPheProGluLeuAsnTyr
GlnLeuLysIleLysValCysIleAspLysAspSerGlyAspValAlaAlaLeuArgGly
SerArgLysPheAsnIleLeuGlyThrAsnThrLysValMetAsnMetGluGluSerAsn
AsnGlySerLeuSerAlaGluPheLysHisLeuThrLeuArgGluGlnArgCysGlyAsn
GlyGlyArgAlaAsnCysAspAlaSerLeuIleValThrGluGluLeuHisLeuIleThr
PheGluThrGluValTyrHisGlnGlyLeuLysIleAspLeuGluThrHisSerLeuPro
ValValValIleSerAsnIleCysGlnMetProAsnAlaTrpAlaSerIleLeuTrpTyr
AsnMetLeuThrAsnAsnProLysAsnValAsnPhePheThrLysProProIleGlyThr
TrpAspGlnValAlaGluValLeuSerTrpGlnPheSerSerThrThrLysArgGlyLeu
SerIleGluGlnLeuThrThrLeuAlaGluLysLeuLeuGlyProGlyValAsnTyrSer
GlyCysGlnIleThrTrpAlaLysPheCysLysGluAsnMetAlaGlyLysGlyPheSer
PheTrpValTrpLeuAspAsnIleIleAspLeuValLysLysTyrIleLeuAlaLeuTrp
AsnGluGlyTyrIleMetGlyPheIleSerLysGluArgGluArgAlaIleLeuSerThr
LysProProGlyThrPheLeuLeuArgPheSerGluSerSerLysGluGlyGlyValThr
PheThrTrpValGluLysAspIleSerGlyLysThrGlnIleGlnSerValGluProTyr
ThrLysGlnGlnLeuAsnAsnMetSerPheAlaGluIleIleMetGlyTyrLysIleMet
AspAlaThrAsnIleLeuValSerProLeuValTyrLeuTyrProAspIleProLysGlu
GluAlaPheGlyLysTyrCysArgProGluSerGlnGluHisProGluAlaAspProGly
SerAlaAlaProTyrLeuLysThrLysPheIleCysValThrProThrThrCysSerAsn
ThrIleAspLeuProMetSerProArgThrLeuAspSerLeuMetGlnPheGlyAsnAsn
GlyGluGlyAlaGluProSerAlaGlyGlyGlnPheGluSerLeuThrPheAspMetGlu
LeuThrSerGluCysAlaThrSerProMet
<210> 11
<211> 828
<212> PRTN
<213> Homo sapiens
<221> CDS
<222> 1-828
<400> 11
MetAspSerGluAlaPheGlnSerAlaArgAspPheLeuAspMetAsnPheGlnSerLeu
AlaMetLysHisMetAspLeuLysGlnMetGluLeuAspThrAlaAlaAlaLysValAsp
GluLeuThrLysGlnLeuGluSerLeuTrpSerAspSerProAlaProProGlyProGln
AlaGlyProProSerArgProProArgTyrSerSerSerSerIleProGluProPheGly
SerArgGlySerProArgLysAlaAlaThrAspGlyAlaAspThrProPheGlyArgSer
GluSerAlaProThrLeuHisProTyrSerProLeuSerProLysGlyArgProSerSer
ProArgThrProLeuTyrLeuGlnProAspAlaTyrGlySerLeuAspArgAlaThrSer
ProArgProArgAlaPheAspGlyAlaGlySerSerLeuGlyArgAlaProSerProArg
ProGlyProGlyProLeuArgGlnGlnGlyProProThrProPheAspPheLeuGlyArg
AlaGlySerProArgGlySerProLeuAlaGluGlyProGlnAlaPhePheProGluArg
GlyProSerProArgProProAlaThrAlaTyrAspAlaProAlaSerAlaPheGlySer
SerLeuLeuGlySerGlyGlySerAlaPheAlaProProLeuArgAlaGlnAspAspLeu
ThrLeuArgArgArgProProLysAlaTrpAsnGluSerAspLeuAspValAlaTyrGlu
LysLysProSerGlnThrAlaSerTyrGluArgLeuAspValPheAlaArgProAlaSer
ProSerLeuGlnLeuLeuProTrpArgGluSerSerLeuAspGlyLeuGlyGlyThrGly
LysAspAsnLeuThrSerAlaThrLeuProArgAsnTyrLysValSerProLeuAlaSer
AspArgArgSerAspAlaGlySerTyrArgArgSerLeuGlySerAlaGlyProSerGly
ThrLeuProArgSerTrpGlnProValSerArgIleProMetProProSerSerProGln
ProArgGlyAlaProArgGlnArgProIleProLeuSerMetIlePheLysLeuGlnAsn
AlaPheTrpGluHisGlyAlaSerArgAlaMetLeuProGlySerProLeuPheThrArg
AlaProProProLysLeuGlnProGlnProGlnProGlnProGlnProGlnSerGlnPro
GlnProGlnLeuProProGlnProGlnThrGlnProGlnThrProThrProAlaProGln
HisProGlnGlnThrTrpProProValAsnGluGlyProProLysProProThrGluLeu
GluProGluProGluIleGluGlyLeuLeuThrProValLeuGluAlaGlyAspValAsp
GluGlyProValAlaArgProLeuSerProThrArgLeuGlnProAlaLeuProProGlu
AlaGlnSerValProGluLeuGluGluValAlaArgValLeuAlaGluIleProArgPro
LeuLysArgArgGlySerMetGluGlnAlaProAlaValAlaLeuProProThrHisLys
LysGlnTyrGlnGlnIleIleSerArgLeuPheHisArgHisGlyGlyProGlyProGly
GlyProGluProGluLeuSerProIleThrGluGlySerGluAlaArgAlaGlyProPro
AlaProAlaProProAlaProIleProProProAlaProSerGlnSerSerProProGlu
GlnProGlnSerMetGluMetArgSerValLeuArgLysAlaGlySerProArgLysAla
ArgArgAlaArgLeuAsnProLeuValLeuLeuLeuAspAlaAlaLeuThrGlyGluLeu
GluValValGlnGlnAlaValLysGluMetAsnAspProSerGlnProAsnGluGluGly
IleThrAlaLeuHisAsnAlaIleCysGlyAlaAsnTyrSerIleValAspPheLeuIle
ThrAlaGlyAlaAsnValAsnSerProAspSerHisGlyTrpThrProLeuHisCysAla
AlaSerCysAsnAspThrValIleCysMetAlaLeuValGlnHisGlyAlaAlaIlePhe
AlaThrThrLeuSerAspGlyAlaThrAlaPheGluLysCysAspProTyrArgGluGly
TyrAlaAspCysAlaThrTyrLeuAlaAspValGluGlnSerMetGlyLeuMetAsnSer
GlyAlaValTyrAlaLeuTrpAspTyrSerAlaGluPheGlyAspGluLeuSerPheArg
GluGlyGluSerValThrValLeuArgArgAspGlyProGluGluThrAspTrpTrpTrp
AlaAlaLeuHisGlyGlnGluGlyTyrValProArgAsnTyrPheGlyLeuPheProArg
ValLysProGlnArgSerLysVal
<210> 12
<211> 364
<212> PRTN
<213> Homo sapiens
<221> CDS
<222> 1-364
<400> 12
MetSerGlyProArgAlaGlyPheTyrArgGlnGluLeuAsnLysThrValTrpGluVal
ProGlnArgLeuGlnGlyLeuArgProValGlySerGlyAlaTyrGlySerValCysSer
AlaTyrAspAlaArgLeuArgGlnLysValAlaValLysLysLeuSerArgProPheGln
SerLeuIleHisAlaArgArgThrTyrArgGluLeuArgLeuLeuLysHisLeuLysHis
GluAsnValIleGlyLeuLeuAspValPheThrProAlaThrSerIleGluAspPheSer
GluValTyrLeuValThrThrLeuMetGlyAlaAspLeuAsnAsnIleValLysCysGln
AlaLeuSerAspGluHisValGlnPheLeuValTyrGlnLeuLeuArgGlyLeuLysTyr
IleHisSerAlaGlyIleIleHisArgAspLeuLysProSerAsnValAlaValAsnGlu
AspCysGluLeuArgIleLeuAspPheGlyLeuAlaArgGlnAlaAspGluGluMetThr
GlyTyrValAlaThrArgTrpTyrArgAlaProGluIleMetLeuAsnTrpMetHisTyr
AsnGlnThrValAspIleTrpSerValGlyCysIleMetAlaGluLeuLeuGlnGlyLys
AlaLeuPheProGlySerAspTyrIleAspGlnLeuLysArgIleMetGluValValGly
ThrProSerProGluValLeuAlaLysIleSerSerGluHisAlaArgThrTyrIleGln
SerLeuProProMetProGlnLysAspLeuSerSerIlePheArgGlyAlaAsnProLeu
AlaIleAspLeuLeuGlyArgMetLeuValLeuAspSerAspGlnArgValSerAlaAla
GluAlaLeuAlaHisAlaTyrPheSerGlnTyrHisAspProGluAspGluProGluAla
GluProTyrAspGluSerValGluAlaLysGluArgThrLeuGluGluTrpLysGluLeu
ThrTyrGlnGluValLeuSerPheLysProProGluProProLysProProGlySerLeu
GluIleGluGln
<210> 13
<211> 358
<212> PRTN
<213> Homo sapiens
<221> CDS
<222> 1-358
<400> 13
MetAsnPheThrValGlyPheLysProLeuLeuGlyAspAlaHisSerMetAspAsnLeu
GluLysGlnLeuIleCysProIleCysLeuGluMetPheSerLysProValValIleLeu
ProCysGlnHisAsnLeuCysArgLysCysAlaAsnAspValPheGlnAlaSerAsnPro
LeuTrpGlnSerArgGlySerThrThrValSerSerGlyGlyArgPheArgCysProSer
CysArgHisGluValValLeuAspArgHisGlyValTyrGlyLeuGlnArgAsnLeuLeu
ValGluAsnIleIleAspIleTyrLysGlnGluSerSerArgProLeuHisSerLysAla
GluGlnHisLeuMetCysGluGluHisGluGluGluLysIleAsnIleTyrCysLeuSer
CysGluValProThrCysSerLeuCysLysValPheGlyAlaHisLysAspCysGluVal
AlaProLeuProThrIleTyrLysArgGlnLysSerGluLeuSerAspGlyIleAlaMet
LeuValAlaGlyAsnAspArgValGlnAlaValIleThrGlnMetGluGluValCysGln
ThrIleGluAspAsnSerArgArgGlnLysGlnLeuLeuAsnGlnArgPheGluSerLeu
CysAlaValLeuGluGluArgLysGlyGluLeuLeuGlnAlaLeuAlaArgGluGlnGlu
GluLysLeuGlnArgValArgGlyLeuIleArgGlnTyrGlyAspHisLeuGluAlaSer
SerLysLeuValGluSerAlaIleGlnSerMetGluGluProGlnMetAlaLeuTyrLeu
GlnGlnAlaLysGluLeuIleAsnLysValGlyAlaMetSerLysValGluLeuAlaGly
ArgProGluProGlyTyrGluSerMetGluGlnPheThrValArgValGluHisValAla
GluMetLeuArgThrIleAspPheGlnProGlyAlaSerGlyGluGluGluGluValAla
ProAspGlyGluGluGlySerAlaGlyProGluGluGluArgProAspGlyPro
<210> 14
<211> 355
<212> PRTN
<213> Homo sapiens
<221> CDS
<222> 1-355
<400> 14
MetProPheLeuGlyGlnAspTrpArgSerProGlyGlnAsnTrpValLysThrAlaAsp
GlyTrpLysArgPheLeuAspGluLysSerGlySerPheValSerAspLeuSerSerTyr
CysAsnLysGluValTyrAsnLysGluAsnLeuPheAsnSerLeuAsnTyrAspValAla
AlaLysLysArgLysLysAspMetLeuAsnSerLysThrLysThrGlnTyrPheHisGln
GluLysTrpIleTyrValHisLysGlySerThrLysGluArgHisGlyTyrCysThrLeu
GlyGluAlaPheAsnArgLeuAspPheSerThrAlaIleLeuAspSerArgArgPheAsn
TyrValValArgLeuLeuGluLeuIleAlaLysSerGlnLeuThrSerLeuSerGlyIle
AlaGlnLysAsnPheMetAsnIleLeuGluLysValValLeuLysValLeuGluAspGln
GlnAsnIleArgLeuIleArgGluLeuLeuGlnThrLeuTyrThrSerLeuCysThrLeu
ValGlnArgValGlyLysSerValLeuValGlyAsnIleAsnMetTrpValTyrArgMet
GluThrIleLeuHisTrpGlnGlnGlnLeuAsnAsnIleGlnIleThrArgProAlaPhe
LysGlyLeuThrPheThrAspLeuProLeuCysLeuGlnLeuAsnIleMetGlnArgLeu
SerAspGlyArgAspLeuValSerLeuGlyGlnAlaAlaProAspLeuHisValLeuSer
GluAspArgLeuLeuTrpLysLysLeuCysGlnTyrHisPheSerGluArgGlnIleArg
LysArgLeuIleLeuSerAspLysGlyGlnLeuAspTrpLysLysMetTyrPheLysLeu
ValArgCysTyrProArgLysGluGlnTyrGlyAspThrLeuGlnLeuCysLysHisCys
HisIleLeuSerTrpLysGlyThrAspHisProCysThrAlaAsnAsnProGluSerCys
SerValSerLeuSerProGlnAspPheIleAsnLeuPheLysPhe
<210> 15
<211> 564
<212> PRTN
<213> Homo sapiens
<221> CDS
<222> 1-564
<400> 15
MetAlaGluLeuAspLeuMetAlaProGlyProLeuProArgAlaThrAlaGlnProPro
AlaProLeuSerProAspSerGlySerProSerProAspSerGlySerAlaSerProVal
GluGluGluAspValGlySerSerGluLysLeuGlyArgGluThrGluGluGlnAspSer
AspSerAlaGluGlnGlyAspProAlaGlyGluGlyLysGluValLeuCysAspPheCys
LeuAspAspThrArgArgValLysAlaValLysSerCysLeuThrCysMetValAsnTyr
CysGluGluHisLeuGlnProHisGlnValAsnIleLysLeuGlnSerHisLeuLeuThr
GluProValLysAspHisAsnTrpArgTyrCysProAlaHisHisSerProLeuSerAla
PheCysCysProAspGlnGlnCysIleCysGlnAspCysCysGlnGluHisSerGlyHis
ThrIleValSerLeuAspAlaAlaArgArgAspLysGluAlaGluLeuGlnCysThrGln
LeuAspLeuGluArgLysLeuLysLeuAsnGluAsnAlaIleSerArgLeuGlnAlaAsn
GlnLysSerValLeuValSerValSerGluValLysAlaValAlaGluMetGlnPheGly
GluLeuLeuAlaAlaValArgLysAlaGlnAlaAsnValMetLeuPheLeuGluGluLys
GluGlnAlaAlaLeuSerGlnAlaAsnGlyIleLysAlaHisLeuGluTyrArgSerAla
GluMetGluLysSerLysGlnGluLeuGluArgMetAlaAlaIleSerAsnThrValGln
PheLeuGluGluTyrCysLysPheLysAsnThrGluAspIleThrPheProSerValTyr
ValGlyLeuLysAspLysLeuSerGlyIleArgLysValIleThrGluSerThrValHis
LeuIleGlnLeuLeuGluAsnTyrLysLysLysLeuGlnGluPheSerLysGluGluGlu
TyrAspIleArgThrGlnValSerAlaValValGlnArgLysTyrTrpThrSerLysPro
GluProSerThrArgGluGlnPheLeuGlnTyrAlaTyrAspIleThrPheAspProAsp
ThrAlaHisLysTyrLeuArgLeuGlnGluGluAsnArgLysValThrAsnThrThrPro
TrpGluHisProTyrProAspLeuProSerArgPheLeuHisTrpArgGlnValLeuSer
GlnGlnSerLeuTyrLeuHisArgTyrTyrPheGluValGluIlePheGlyAlaGlyThr
TyrValGlyLeuThrCysLysGlyIleAspArgLysGlyGluGluArgAsnSerCysIle
SerGlyAsnAsnPheSerTrpSerLeuGlnTrpAsnGlyLysGluPheThrAlaTrpTyr
SerAspMetGluThrProLeuLysAlaGlyProPheArgArgLeuGlyValTyrIleAsp
PheProGlyGlyIleLeuSerPheTyrGlyValGluTyrAspThrMetThrLeuValHis
LysPheAlaCysLysPheSerGluProValTyrAlaAlaPheTrpLeuSerLysLysGlu
AsnAlaIleArgIleValAspLeuGlyGluGluProGluLysProAlaProSerLeuVal
GlyThrAlaPro
<210> 16
<211> 440
<212> PRTN
<213> Homo sapiens
<221> CDS
<222> 1-440
<400> 16
MetAlaSerLeuThrValLysAlaTyrLeuLeuGlyLysGluAspAlaAlaArgGluIle
ArgArgPheSerPheCysCysSerProGluProGluAlaGluAlaGluAlaAlaAlaGly
ProGlyProCysGluArgLeuLeuSerArgValAlaAlaLeuPheProAlaLeuArgPro
GlyGlyPheGlnAlaHisTyrArgAspGluAspGlyAspLeuValAlaPheSerSerAsp
GluGluLeuThrMetAlaMetSerTyrValLysAspAspIlePheArgIleTyrIleLys
GluLysLysGluCysArgArgAspHisArgProProCysAlaGlnGluAlaProArgAsn
MetValHisProAsnValIleCysAspGlyCysAsnGlyProValValGlyThrArgTyr
LysCysSerValCysProAspTyrAspLeuCysSerValCysGluGlyLysGlyLeuHis
ArgGlyHisThrLysLeuAlaPheProSerProPheGlyHisLeuSerGluGlyPheSer
HisSerArgTrpLeuArgLysValLysHisGlyHisPheGlyTrpProGlyTrpGluMet
GlyProProGlyAsnTrpSerProArgProProArgAlaGlyGluAlaArgProGlyPro
ThrAlaGluSerAlaSerGlyProSerGluAspProSerValAsnPheLeuLysAsnVal
GlyGluSerValAlaAlaAlaLeuSerProLeuGlyIleGluValAspIleAspValGlu
HisGlyGlyLysArgSerArgLeuThrProValSerProGluSerSerSerThrGluGlu
LysSerSerSerGlnProSerSerCysCysSerAspProSerLysProGlyGlyAsnVal
GluGlyAlaThrGlnSerLeuAlaGluGlnMetArgLysIleAlaLeuGluSerGluGly
ArgProGluGluGlnMetGluSerAspAsnCysSerGlyGlyAspAspAspTrpThrHis
LeuSerSerLysGluValAspProSerThrGlyGluLeuGlnSerLeuGlnMetProGlu
SerGluGlyProSerSerLeuAspProSerGlnGluGlyProThrGlyLeuLysGluAla
AlaLeuTyrProHisLeuProProGluAlaAspProArgLeuIleGluSerLeuSerGln
MetLeuSerMetGlyPheSerAspGluGlyGlyTrpLeuThrArgLeuLeuGlnThrLys
AsnTyrAspIleGlyAlaAlaLeuAspThrIleGlnTyrSerLysHisProProProLeu
<210> 17
<211> 465
<212> PRTN
<213> Homo sapiens
<221> CDS
<222> 1-465
<400> 17
MetIleValPheValArgPheAsnSerSerHisGlyPheProValGluValAspSerAsp
ThrSerIlePheGlnLeuLysGluValValAlaLysArgGlnGlyValProAlaAspGln
LeuArgValIlePheAlaGlyLysGluLeuArgAsnAspTrpThrValGlnAsnCysAsp
LeuAspGlnGlnSerIleValHisIleValGlnArgProTrpArgLysGlyGlnGluMet
AsnAlaThrGlyGlyAspAspProArgAsnAlaAlaGlyGlyCysGluArgGluProGln
SerLeuThrArgValAspLeuSerSerSerValLeuProGlyAspSerValGlyLeuAla
ValIleLeuHisThrAspSerArgLysAspSerProProAlaGlySerProAlaGlyArg
SerIleTyrAsnSerPheTyrValTyrCysLysGlyProCysGlnArgValGlnProGly
LysLeuArgValGlnCysSerThrCysArgGlnAlaThrLeuThrLeuThrGlnGlyPro
SerCysTrpAspAspValLeuIleProAsnArgMetSerGlyGluCysGlnSerProHis
CysProGlyThrSerAlaGluPhePhePheLysCysGlyAlaHisProThrSerAspLys
GluThrSerValAlaLeuHisLeuIleAlaThrAsnSerArgAsnIleThrCysIleThr
CysThrAspValArgSerProValLeuValPheGlnCysAsnSerArgHisValIleCys
LeuAspCysPheHisLeuTyrCysValThrArgLeuAsnAspArgGlnPheValHisAsp
ProGlnLeuGlyTyrSerLeuProCysValAlaGlyCysProAsnSerLeuIleLysGlu
LeuHisHisPheArgIleLeuGlyGluGluGlnTyrAsnArgTyrGlnGlnTyrGlyAla
GluGluCysValLeuGlnMetGlyGlyValLeuCysProArgProGlyCysGlyAlaGly
LeuLeuProGluProAspGlnArgLysValThrCysGluGlyGlyAsnGlyLeuGlyCys
GlyPheAlaPheCysArgGluCysLysGluAlaTyrHisGluGlyGluCysSerAlaVal
PheGluAlaSerGlyThrThrThrGlnAlaTyrArgValAspGluArgAlaAlaGluGln
AlaArgTrpGluAlaAlaSerLysGluThrIleLysLysThrThrLysProCysProArg
CysHisValProValGluLysAsnGlyGlyCysMetHisMetLysCysProGlnProGln
CysArgLeuGluTrpCysTrpAsnCysGlyCysGluTrpAsnArgValCysMetGlyAsp
HisTrpPheAspVal
<210> 18
<211> 194
<212> PRTN
<213> Homo sapiens
<221> CDS
<222> 1-194
<400> 18
MetSerGlnAsnGlyAlaProGlyMetGlnGluGluSerLeuGlnGlySerTrpValGlu
LeuHisPheSerAsnAsnGlyAsnGlyGlySerValProAlaSerValSerIleTyrAsn
GlyAspMetGluLysIleLeuLeuAspAlaGlnHisGluSerGlyArgSerSerSerLys
SerSerHisCysAspSerProProArgSerGlnThrProGlnAspThrAsnArgAlaSer
GluThrAspThrHisSerIleGlyGluLysAsnSerSerGlnSerGluGluAspAspIle
GluArgArgLysGluValGluSerIleLeuLysLysAsnSerAspTrpIleTrpAspTrp
SerSerArgProGluAsnIleProProLysGluPheLeuPheLysHisProLysArgThr
AlaThrLeuSerMetArgAsnThrSerValMetLysLysGlyGlyIlePheSerAlaGlu
PheLeuLysValPheLeuProSerLeuLeuLeuSerHisLeuLeuAlaIleGlyLeuGly
IleTyrIleGlyArgArgLeuThrThrSerThrSerThrPhe
<210> 19
<211> 283
<212> PRTN
<213> Homo sapiens
<221> CDS
<222> 1-283
<400> 19
MetAlaValProProThrTyrAlaAspLeuGlyLysSerAlaArgAspValPheThrLys
GlyTyrGlyPheGlyLeuIleLysLeuAspLeuLysThrLysSerGluAsnGlyLeuGlu
PheThrSerSerGlySerAlaAsnThrGluThrThrLysValThrGlySerLeuGluThr
LysTyrArgTrpThrGluTyrGlyLeuThrPheThrGluLysTrpAsnThrAspAsnThr
LeuGlyThrGluIleThrValGluAspGlnLeuAlaArgGlyLeuLysLeuThrPheAsp
SerSerPheSerProAsnThrGlyLysLysAsnAlaLysIleLysThrGlyTyrLysArg
GluHisIleAsnLeuGlyCysAspMetAspPheAspIleAlaGlyProSerIleArgGly
AlaLeuValLeuGlyTyrGluGlyTrpLeuAlaGlyTyrGlnMetAsnPheGluThrAla
LysSerArgValThrGlnSerAsnPheAlaValGlyTyrLysThrAspGluPheGlnLeu
HisThrAsnValAsnAspGlyThrGluPheGlyGlySerIleTyrGlnLysValAsnLys
LysLeuGluThrAlaValAsnLeuAlaTrpThrAlaGlyAsnSerAsnThrArgPheGly
IleAlaAlaLysTyrGlnIleAspProAspAlaCysPheSerAlaLysValAsnAsnSer
SerLeuIleGlyLeuGlyTyrThrGlnThrLeuLysProGlyIleLysLeuThrLeuSer
AlaLeuLeuAspGlyLysAsnValAsnAlaGlyGlyHisLysLeuGlyLeuGlyLeuGlu
PheGlnAla
<210> 20
<211> 105
<212> PRTN
<213> Homo sapiens
<221> CDS
<222> 1-105
<400> 20
MetGlyAspValGluLysGlyLysLysIlePheIleMetLysCysSerGlnCysHisThr
ValGluLysGlyGlyLysHisLysThrGlyProAsnLeuHisGlyLeuPheGlyArgLys
ThrGlyGlnAlaProGlyTyrSerTyrThrAlaAlaAsnLysAsnLysGlyIleIleTrp
GlyGluAspThrLeuMetGluTyrLeuGluAsnProLysLysTyrIleProGlyThrLys
MetIlePheValGlyIleLysLysLysGluGluArgAlaAspLeuIleAlaTyrLeuLys
LysAlaThrAsnGlu
<210> 21
<211> 260
<212> PRTN
<213> Homo sapiens
<221> CDS
<222> 1-260
<400> 21
MKIKNFFLNFNRKSVQNNNSLNHFDLSYRVFRIYRWNPSIMKNPWINSYI
VSMNNCGPIVLDALIKIKNEQDSTLTFRRSCREGICGSCAINIIGKNTLACLYKLSDSSR
FIHIYPLPHIYVIKDLVPDLTNFYTQHKSIQPWLQPNRDYSLQKEFIQSKQERSYLDGL
YECILCACCSASCPSYWWNGDRYLGPAILLQAYRWIIDSRDSATEKRLGYLNDSYKV
YRCHTIMNCTKTCPKHLNPGKAIANIKILLKGEK
<210> 22
<211> 81
<212> PRTN
<213> Homo sapiens
<221> CDS
<222> 1-81
<400> 22
MLRQIIGQAKKHPSLIPLFVFIGTGATGATLYLLRLALFNPDVCWDRNNPE
PWNKLGPNDQYKFYSVNVDYSKLKKERPDF
<210> 23
<211> 553
<212> PRTN
<213> Homo sapiens
<221> CDS
<222> 1-51
<400> 23
MLSVRVAAAVVRALPRRAGLVSRNALGSSFIAARNFHASNTHLQKTGTA
EMSSILEERILGADTSVDLEETGRVLSIGDGIARVHGLRNVQAEEMVEFSSGLKGMSL
NLEPDNVGVVVFGNDKLIKEGDIVKRTGAIVDVPVGEELLGRVVDALGNAIDGKGPI
GSKTRRRVGLKAPGIIPRISVREPMQTGIKAVDSLVPIGRGQRELIIGDRQTGKTSIAID
TIINQKRENDGSDEKKKLYCIYVAIGQKRSTVAQLVKRLTDADAMKYTIVVSATASD
AAPLQYLAPYSGCSMGEYFRDNGKHALIIYDDLSKQAVAYRQMSLLLRRPPGREAY
PGDVFYLHSRLLERAAKMNDAFGGGSLTALPVIETQAGDVSAYIPTNVISITDGQIFL
ETELFYKGIRPAINVGLSVSRVGSAAQTRAMKQVAGTMKLELAQYREVAAFAQFGS
DLDAATQQLLSRGVRLTELLKQGQYSPMAIEEQVAVIYAGVRGYLDKLEPSKITKFE
NAFLSHVVSQHQALLGTIRADGKISEQSDAKLKEIVINFLAGFEA
<210> 24
<211> 727
<212> PRTN
<213> Homo sapiens
<221> CDS
<222> 1-727
<400> 24
MLRIPVRKALVGLSKSPKGCVRTTATAASNLIEVFVDGQSVMVEPGTTVL
QACEKVGMQIPRFCYHERLSVAGNCRMCLVEIEKAPKVVAACAMPVMKGWNILIN
SEKSKKAREGVMEFLLANHPLDCPICDQGGECDLQDQSMMFGNDRSRFLEGKRAVE
DKNIGPLVKTIMTRCIQCTRCIRFASEIAGVDDLGTTGRGNDMQVGTYIEKMFMSELS
GNIIDICPVGALTSKPYAFTARPWETRKTESIDVMDAVGSNIVVSTRTGEVMRILPRM
HEDINEEWISDKTRFAYDGLKRQRLTEPMVRNEKGLLTYTSWEDALSRVAGMLQSF
QGKDVAAIAGGLVDAEALVALKDLLNRVDSDTLCTEEVFPTAGAGTDLRSNYLLNT
TIAGVEEADVVLLVGTNPRFEAPLFNARIRKSWLHNDLKVALIGSPVDLTYTYDHLG
DSPKILQDIASGSHPFSQVLKEAKKPMVVLGSSALQRNDGAAILAAVSSIAQKIRMTS
GVTGDWKVMNILHRIASQVAALDLGYKPGVEAIRKNPPKVLFLLGADGGCITRQDL
PKDCFIIYQGHHGDVGAPIADVILPGAAYTEKSATYVNTEGRAQQTKVAVTPPGLAR
EDWKIIRALSEIAGMTLPYDTLDQVRNRLEEVSPNLVRYDDIEGANYFQQANELSKL
VNQQLLADPLVPPQLTIKDFYMTDSISRASQTMAKCVKAVTEGAQAVEEPSIC
<210> 25
<211> 81
<212> PRTN
<213> Homo sapiens
<221> CDS
<222> 1-81
<400> 25
MLRQIIGQAKKHPSLIPLFVFIGTGATGATLYLLRLALFNPDVCWDRNNPE
PWNKLGPNDQYKFYSVNVDYSKLKKERPDF

What is claimed is:

1. A method of treatment for protecting against muscle wasting induced muscle mass loss comprising:
administering a therapeutically effective amount of at least one polyphenol flavonoid to a subject;
wherein administration of the at least one polyphenol flavonoid protects against muscle wasting via increasing mitochondrial biogenesis; and
administering the at least one polyphenol flavonoid increases muscle mass and muscle cross section in the subject.

2. The method of treatment of claim 1, wherein administration of the at least one polyphenol flavonoid prevents or treats Cachexia or sarcopenia.

3. The method of treatment of claim 1, wherein administration of the at least one polyphenol flavonoid prevents or treats Cachexia induced by chronic obstructive pulmonary disease, heart failure, chronic kidney disease, HIV, at least one cancer, chemotherapy, other chronic disease treatment, or combinations of the above.

4. The method of treatment of claim 1, wherein the subject undergoing administration of the at least one polyphenol flavonoid has increased expression of SEQ ID NO: 1 and reduced expression of SEQ ID NO: 2 in the subject as compared to prior to treatment with the method.

5. The method of treatment of claim 1, wherein the at least one polyphenol flavonoid comprises 3,4,5,7-pentahydroxylflavone (quercetin).

6. The method of treatment of claim 1, wherein administration of the at least one polyphenol flavonoid equilibrates molecular signaling to protect against mitochondrial dysfunction.

7. The method of treatment of claim 1, wherein administration of the at least one polyphenol flavonoid increased expression of SEQ ID NO: 18 and decreased expression of SEQ ID NO: 6 and SEQ ID NO: 7 in the subject as compared to prior to treatment with the method.

8. The method of treatment of claim 1, wherein administration of the at least one polyphenol flavonoid produces anti-cachectic properties demonstrated by preserving muscle mass and muscle cross sectional area with improved mitochondrial content in the subject.

9. The method of treatment of claim 1, wherein administration of the at least one polyphenol flavonoid increases a number of intermyofibrillar mitochondria and increases size of the intermyofibrillar mitochondria.

10. The method of treatment of claim 1, wherein administration of the at least one polyphenol flavonoid is provided to the subject bearing at least one tumor.

11. The method of treatment of claim 1, wherein administration of the at least one polyphenol flavonoid is administered with 5-fluorouracil.

12. The method of treatment of claim 11, wherein administration of the at least one polyphenol flavonoid does not reduce tumor suppressive capability of 5-fluorouracil.

13. The method of treatment of claim 1, wherein administration of the at least one polyphenol flavonoid increases succinate dehydrogenase activity in at least one myofibrillar fiber.

14. The method of treatment of claim 1, wherein administration of the at least one polyphenol flavonoid increased expression of SEQ ID NO: 19 and increased expression of SEQ ID NO: 20 in the subject as compared to prior to treatment with the method.

15. The method of treatment of claim 1, wherein administration of the at least one polyphenol flavonoid increased expression of SEQ ID NO: 17 and increased expression of SEQ ID NO: 18 in the subject as compared to prior to treatment with the method.

16. The method of treatment of claim 1, wherein administration of the at least one polyphenol flavonoid reduced expression of SEQ ID NO: 6 and reduced expression of SEQ ID NO: 7 in the subject as compared to prior to treatment with the method.

17. The method of treatment of claim 1, wherein administration of the at least one polyphenol flavonoid equilibrates molecular signaling to protect against mitochondrial dysfunction.


SEQUENCE LISTING (25 sequences)

SEQ ID NO: 1 - protein, 741 aa, Homo sapiens
MAEPVSPLKHFVLAKKAITAIFDQLLEFVTEGSHFVEATYKNPELDRIATEDDLVEMQGYKDKLSIIGEVLSRRHMKVAFFGRTSSGKSSVINAMLWDKVLPSGIGHITNCFLSVEGTDGDKAYLMTEGSDEKKSVKTVNQLAHALHMDKDLKAGCLVRVFWPKAKCALLRDDLVLVDSPGTDVTTELDSWIDKFCLDADVFVLVANSESTLMNTEKHFFHKVNERLSKPNIFILNNRWDASASEPEYMEDVRRQHMERCLHFLVEELKVVNALEAQNRIFFVSAKEVLSARKQKAQGMPESGVALAEGFHARLQEFQNFEQIFEECISQSAVKTKFEQHTIRAKQILATVKNIMDSVNLAAEDKRHYSVEEREDQIDRLDFIRNQMNLLTLDVKKKIKEVTEEVANKVSCAMTDEICRLSVLVDEFCSEFHPNPDVLKIYKSELNKHIEDGMGRNLADRCTDEVNALVLQTQQEIIENLKPLLPAGIQDKLHTLIPCKKFDLSYNLNYHKLCSDFQEDIVFRFSLGWSSLVHRFLGPRNAQRVLLGLSEPIFQLPRSLASTPTAPTTPATPDNASQEELMITLVTGLASVTSRTSMGIIIVGGVIWKTIGWKLLSVSLTMYGALYLYERLSWTTHAKERAFKQQFVNYATEKLRMIVSSTSANCSHQVKQQIATTFARLCQQVDITQKQLEEEIARLPKEIDQLEKIQNNSKLLRNKAVQLENELENFTKQFLPSSNEES

SEQ ID NO: 2 - protein, 292 aa, Homo sapiens
MNPFWSMSTSSVRKRSEGEEKTLTGDVKTSPPRTAPKKQLPSIPKNALPITKPTSPAPAAQSTNGTHASYGPFYLEYSLLAEFTLVVKQKLPGVYVQPSYRSALMWFGVIFIRHGLYQDGVFKFTVYIPDNYPDGDCPRLVFDIPVFHPLVDPTSGELDVKRAFAKWRRNHNHIWQVLMYARRVFYKIDTASPLNPEAAVLYEKDIQLFKSKVVDSVKVCTARLFDQPKIEDPYAISFSPWNPSVHDEAREKMLTQKKPEEQHNKSVHVAGLSWVKPGSVQPFSKEEKTVAT

SEQ ID NO: 3 - protein, 757 aa, Homo sapiens
MSLLFSRCNSIVTVKKNKRHMAEVNASPLKHFVTAKKKINGIFEQLGAYIQESATFLEDTYRNAELDPVTTEEQVLDVKGYLSKVRGISEVLARRHMKVAFFGRTSNGKSTVINAMLWDKVLPSGIGHTTNCFLRVEGTDGHEAFLLTEGSEEKRSAKTVNQLAHALHQDKQLHAGSLVSVMWPNSKCPLLKDDLVLMDSPGIDVTTELDSWIDKFCLDADVFVLVANSESTLMQTEKHFFHKVSERLSRPNIFILNNRWDASASEPEYMEEVRRQHMERCTSFLVDELGVVDRSQAGDRIFFVSAKEVLNARIQKAQGMPEGGGALAEGFQVRMFEFQNFERRFEECISQSAVKTKFEQHTVRAKQIAEAVRLIMDSLHMAAREQQVYCEEMREERQDRLKFIDKQLELLAQDYKLRIKQITEEVERQVSTAMAEEIRRLSVLVDDYQMDFHPSPVVLKVYKNELHRHIEEGLGRNMSDRCSTAITNSLQTMQQDMIDGLKPLLPVSVRSQIDMLVPRQCFSLNYDLNCDKLCADFQEDIEFHFSLGWTMLVNRFLGPKNSRRALMGYNDQVQRPIPLTPANPSMPPLPQGSLTQEEFMVSMVTGLASLTSRTSMGILVVGGVVWKAVGWRLIALSFGLYGLLYVYERLTWTTKAKERAFKRQFVEHASEKLQLVISYTGSNCSHQVQQELSGTFAHLCQQVDVTRENLEQEIAAMNKKIEVLDSLQSKAKLLRNKAGWLDSELNMFTHQYLQPSR

SEQ ID NO: 4 - protein, 960 aa, Homo sapiens
MWRLRRAAVACEVCQSLVKHSSGIKGSLPLQKLHLVSRSIYHSHHPTLKLQRPQLRTSFQQFSSLTNLPLRKLKFSPIKYGYQPRRNFWPARLATRLLKLRYLILGSAVGGGYTAKKTFDQWKDMIPDLSEYKWIVPDIVWEIDEYIDFEKIRKALPSSEDLVKLAPDFDKIVESLSLLKDFFTSGSPEETAFRATDRGSESDKHFRKVSDKEKIDQLQEELLHTQLKYQRILERLEKENKELRKLVLQKDDKGIHHRKLKKSLIDMYSEVLDVLSDYDASYNTQDHLPRVVVVGDQSAGKTSVLEMIAQARIFPRGSGEMMTRSPVKVTLSEGPHHVALFKDSSREFDLTKEEDLAALRHEIELRMRKNVKEGCTVSPETISLNVKGPGLQRMVLVDLPGVINTVTSGMAPDTKETIFSISKAYMQNPNAIILCIQDGSVDAERSIVTDLVSQMDPHGRRTIFVLTKVDLAEKNVASPSRIQQIIEGKLFPMKALGYFAVVTGKGNSSESIEAIREYEEEFFQNSKLLKTSMLKAHQVTTRNLSLAVSDCFWKMVRESVEQQADSFKATRFNLETEWKNNYPRLRELDRNELFEKAKNEILDEVISLSQVTPKHWEEILQQSLWERVSTHVIENIYLPAAQTMNSGTFNTTVDIKLKQWTDKQLPNKAVEVAWETLQEEFSRFMTEPKGKEHDDIFDKLKEAVKEESIKRHKWNDFAEDSLRVIQHNALEDRSISDKQQWDAAIYFMEEALQARLKDTENAIENMVGPDWKKRWLYWKNRTQEQCVHNETKNELEKMLKCNEEHPAYLASDEITTVRKNLESRGVEVDPSLIKDTWHQVYRRHFLKTALNHCNLCRRGFYYYQRHFVDSELECNDVVLFWRIQRMLAITANTLRQQLTNTEVRRLEKNVKEVLEDFAEDGEKKIKLLTGKRVQLAEDLKKVREIQEKLDAFIEALHQEK

SEQ ID NO: 5 - protein, 198 aa, Homo sapiens
MAADISESSGADCKGDPRNSAKLDADYPLRVLYCGVCSLPTEYCEYMPDVAKCRQWLEKNFPNEFAKLTVENSPKQEAGISEGQGTAGEEEEKKKQKRGGRGQIKQKKKTVPQKVTIAKIPRAKKKYVTRVCGLATFEIDLKEAQRFFAQKFSCGASVTGEDEIIIQGDFTDDIIDVIQEKWPEVDDDSIEDLGEVKK

SEQ ID NO: 6 - protein, 246 aa, Homo sapiens
MAFLRSMWGVLSALGRSGAELCTGCGSRLRSPFSFVYLPRWFSSVLASCPKKPVSSYLRFSKEQLPIFKAQNPDAKTTELIRRIAQRWRELPDSKKKIYQDAYRAEWQVYKEEISRFKEQLTPSQIMSLEKEIMDKHLKRKAMTKKKELTLLGKPKRPRSAYNVYVAERFQEAKGDSPQEKLKTVKENWKNLSDSEKELYIQHAKEDETRYHNEMKSWEEQMIEVGRKDLLRRTIKKQRKYGAEEC

SEQ ID NO: 7 - protein, 505 aa, Homo sapiens
MGETLGDSPIDPESDSFTDTLSANISQEMTMVDTEMPFWPTNFGISSVDLSVMEDHSHSFDIKPFTTVDFSSISTPHYEDIPFTRTDPVVADYKYDLKLQEYQSAIKVEPASPPYYSEKTQLYNKPHEEPSNSLMAIECRVCGDKASGFHYGVHACEGCKGFFRRTIRLKLIYDRCDLNCRIHKKSRNKCQYCRFQKCLAVGMSHNAIRFGRMPQAEKEKLLAEISSDIDQLNPESADLRALAKHLYDSYIKSFPLTKAKARAILTGKTTDKSPFVIYDMNSLMMGEDKIKFKHITPLQEQSKEVAIRIFQGCQFRSVEAVQEITEYAKSIPGFVNLDLNDQVTLLKYGVHEIIYTMLASLMNKDGVLISEGQGFMTREFLKSLRKPFGDFMEPKFEFAVKFNALELDDSDLAIFIAVIILSGDRPGLLNVKPIEDIQDNLLQALELQLKLNHPESSQLFAKLLQKMTDLRQIVTEHVQLLQVIKKTETDMSLHPLLQEIYKDLY

SEQ ID NO: 8 - protein, 798 aa, Homo sapiens
MAWDMCNQDSESVWSDIECAALVGEDQPLCPDLPELDLSELDVNDLDTDSFLGGLKWCSDQSEIISNQYNNEPSNIFEKIDEENEANLLAVLTETLDSLPVDEDGLPSFDALTDGDVTTDNEASPSSMPDGTPPPQEAEEPSLLKKLLLAPANTQLSYNECSGLSTQNHANHNHRIRTNPAIVKTENSWSNKAKSICQQQKPQRRPCSELLKYLTTNDDPPHTKPTENRNSSRDKCTSKKKSHTQSQSQHLQAKPTTLSLPLTPESPNDPKGSPFENKTIERTLSVELSGTAGLTPPTTPPHKANQDNPFRASPKLKSSCKTVVPPPSKKPRYSESSGTQGNNSTKKGPEQSELYAQLSKSSVLTGGHEERKTKRPSLRLFGDHDYCQSINSKTEILINISQELQDSRQLENKDVSSDWQGQICSSTDSDQCYLRETLEASKQVSPCSTRKQLQDQEIRAELNKHFGHPSQAVFDDEADKTGELRDSDFSNEQFSKLPMFINSGLAMDGLFDDSEDESDKLSYPWDGTQSYSLFNVSPSCSSFNSPCRDSVSPPKSLFSQRPQRMRSRSRSFSRHRSCSRSPYSRSRSRSPGSRSSSRSCYYYESSHYRHRTHRNSPLYVRSRSRSPYSRRPRYDSYEEYQHERLKREEYRREYEKRESERAKQRERQRQKAIEERRVIYVGKIRPDTTRTELRDRFEVFGEIEECTVNLRDDGDSYGFITYRYTCDAFAALENGYTLRRSNETDFELYFCGRKQFFKSNYADLDSNSDDFDPASTKSKYDSLDFDSLLKEAQRSLRR

SEQ ID NO: 9 - protein, 212 aa, Homo sapiens
MNSFSTSAFGPVAFSLGLLLVLPAAFPAPVPPGEDSKDVAAPHRQPLTSSERIDKQIRYILDGISALRKETCNKSNMCESSKEALAENNLNLPKMAEKDGCFQSGFNEETCLVKIITGLLEFEVYLEYLQNRFESSEEQARAVQMSTKVLIQFLQKKAKNLDAITTPDPTTNASLLTKLQAQNQWLQDMTTHLILRSFKEFLQSSLRALRQM

SEQ ID NO: 10 - protein, 770 aa, Homo sapiens
MAQWNQLQQLDTRYLEQLHQLYSDSFPMELRQFLAPWIESQDWAYAASKESHATLVFHNLLGEIDQQYSRFLQESNVLYQHNLRRIKQFLQSRYLEKPMEIARIVARCLWEESRLLQTAATAAQQGGQANHPTAAVVTEKQQMLEQHLQDVRKRVQDLEQKMKVVENLQDDFDFNYKTLKSQGDMQDLNGNNQSVTRQKMQQLEQMLTALDQMRRSIVSELAGLLSAMEYVQKTLTDEELADWKRRQQIACIGGPPNICLDRLENWITSLAESQLQTRQQIKKLEELQQKVSYKGDPIVQHRPMLEERIVELFRNLMKSAFVVERQPCMPMHPDRPLVIKTGVQFTTKVRLLVKFPELNYQLKIKVCIDKDSGDVAALRGSRKFNILGTNTKVMNMEESNNGSLSAEFKHLTLREQRCGNGGRANCDASLIVTEELHLITFETEVYHQGLKIDLETHSLPVVVISNICQMPNAWASILWYNMLTNNPKNVNFFTKPPIGTWDQVAEVLSWQFSSTTKRGLSIEQLTTLAEKLLGPGVNYSGCQITWAKFCKENMAGKGFSFWVWLDNIIDLVKKYILALWNEGYIMGFISKERERAILSTKPPGTFLLRFSESSKEGGVTFTWVEKDISGKTQIQSVEPYTKQQLNNMSFAEIIMGYKIMDATNILVSPLVYLYPDIPKEEAFGKYCRPESQEHPEADPGSAAPYLKTKFICVTPTTCSNTIDLPMSPRTLDSLMQFGNNGEGAEPSAGGQFESLTFDMELTSECATSPM

SEQ ID NO: 11 - protein, 828 aa, Homo sapiens
MDSEAFQSARDFLDMNFQSLAMKHMDLKQMELDTAAAKVDELTKQLESLWSDSPAPPGPQAGPPSRPPRYSSSSIPEPFGSRGSPRKAATDGADTPFGRSESAPTLHPYSPLSPKGRPSSPRTPLYLQPDAYGSLDRATSPRPRAFDGAGSSLGRAPSPRPGPGPLRQQGPPTPFDFLGRAGSPRGSPLAEGPQAFFPERGPSPRPPATAYDAPASAFGSSLLGSGGSAFAPPLRAQDDLTLRRRPPKAWNESDLDVAYEKKPSQTASYERLDVFARPASPSLQLLPWRESSLDGLGGTGKDNLTSATLPRNYKVSPLASDRRSDAGSYRRSLGSAGPSGTLPRSWQPVSRIPMPPSSPQPRGAPRQRPIPLSMIFKLQNAFWEHGASRAMLPGSPLFTRAPPPKLQPQPQPQPQPQSQPQPQLPPQPQTQPQTPTPAPQHPQQTWPPVNEGPPKPPTELEPEPEIEGLLTPVLEAGDVDEGPVARPLSPTRLQPALPPEAQSVPELEEVARVLAEIPRPLKRRGSMEQAPAVALPPTHKKQYQQIISRLFHRHGGPGPGGPEPELSPITEGSEARAGPPAPAPPAPIPPPAPSQSSPPEQPQSMEMRSVLRKAGSPRKARRARLNPLVLLLDAALTGELEVVQQAVKEMNDPSQPNEEGITALHNAICGANYSIVDFLITAGANVNSPDSHGWTPLHCAASCNDTVICMALVQHGAAIFATTLSDGATAFEKCDPYREGYADCATYLADVEQSMGLMNSGAVYALWDYSAEFGDELSFREGESVTVLRRDGPEETDWWWAALHGQEGYVPRNYFGLFPRVKPQRSKV

SEQ ID NO: 12 - protein, 364 aa, Homo sapiens
MSGPRAGFYRQELNKTVWEVPQRLQGLRPVGSGAYGSVCSAYDARLRQKVAVKKLSRPFQSLIHARRTYRELRLLKHLKHENVIGLLDVFTPATSIEDFSEVYLVTTLMGADLNNIVKCQALSDEHVQFLVYQLLRGLKYIHSAGIIHRDLKPSNVAVNEDCELRILDFGLARQADEEMTGYVATRWYRAPEIMLNWMHYNQTVDIWSVGCIMAELLQGKALFPGSDYIDQLKRIMEVVGTPSPEVLAKISSEHARTYIQSLPPMPQKDLSSIFRGANPLAIDLLGRMLVLDSDQRVSAAEALAHAYFSQYHDPEDEPEAEPYDESVEAKERTLEEWKELTYQEVLSFKPPEPPKPPGSLEIEQ

SEQ ID NO: 13 - protein, 358 aa, Homo sapiens
MNFTVGFKPLLGDAHSMDNLEKQLICPICLEMFSKPVVILPCQHNLCRKCANDVFQASNPLWQSRGSTTVSSGGRFRCPSCRHEVVLDRHGVYGLQRNLLVENIIDIYKQESSRPLHSKAEQHLMCEEHEEEKINIYCLSCEVPTCSLCKVFGAHKDCEVAPLPTIYKRQKSELSDGIAMLVAGNDRVQAVITQMEEVCQTIEDNSRRQKQLLNQRFESLCAVLEERKGELLQALAREQEEKLQRVRGLIRQYGDHLEASSKLVESAIQSMEEPQMALYLQQAKELINKVGAMSKVELAGRPEPGYESMEQFTVRVEHVAEMLRTIDFQPGASGEEEEVAPDGEEGSAGPEEERPDGP

SEQ ID NO: 14 - protein, 355 aa, Homo sapiens
MPFLGQDWRSPGQNWVKTADGWKRFLDEKSGSFVSDLSSYCNKEVYNKENLFNSLNYDVAAKKRKKDMLNSKTKTQYFHQEKWIYVHKGSTKERHGYCTLGEAFNRLDFSTAILDSRRFNYVVRLLELIAKSQLTSLSGIAQKNFMNILEKVVLKVLEDQQNIRLIRELLQTLYTSLCTLVQRVGKSVLVGNINMWVYRMETILHWQQQLNNIQITRPAFKGLTFTDLPLCLQLNIMQRLSDGRDLVSLGQAAPDLHVLSEDRLLWKKLCQYHFSERQIRKRLILSDKGQLDWKKMYFKLVRCYPRKEQYGDTLQLCKHCHILSWKGTDHPCTANNPESCSVSLSPQDFINLFKF

SEQ ID NO: 15 - protein, 564 aa, Homo sapiens
MAELDLMAPGPLPRATAQPPAPLSPDSGSPSPDSGSASPVEEEDVGSSEKLGRETEEQDSDSAEQGDPAGEGKEVLCDFCLDDTRRVKAVKSCLTCMVNYCEEHLQPHQVNIKLQSHLLTEPVKDHNWRYCPAHHSPLSAFCCPDQQCICQDCCQEHSGHTIVSLDAARRDKEAELQCTQLDLERKLKLNENAISRLQANQKSVLVSVSEVKAVAEMQFGELLAAVRKAQANVMLFLEEKEQAALSQANGIKAHLEYRSAEMEKSKQELERMAAISNTVQFLEEYCKFKNTEDITFPSVYVGLKDKLSGIRKVITESTVHLIQLLENYKKKLQEFSKEEEYDIRTQVSAVVQRKYWTSKPEPSTREQFLQYAYDITFDPDTAHKYLRLQEENRKVTNTTPWEHPYPDLPSRFLHWRQVLSQQSLYLHRYYFEVEIFGAGTYVGLTCKGIDRKGEERNSCISGNNFSWSLQWNGKEFTAWYSDMETPLKAGPFRRLGVYIDFPGGILSFYGVEYDTMTLVHKFACKFSEPVYAAFWLSKKENAIRIVDLGEEPEKPAPSLVGTAP

SEQ ID NO: 16 - protein, 440 aa, Homo sapiens
MASLTVKAYLLGKEDAAREIRRFSFCCSPEPEAEAEAAAGPGPCERLLSRVAALFPALRPGGFQAHYRDEDGDLVAFSSDEELTMAMSYVKDDIFRIYIKEKKECRRDHRPPCAQEAPRNMVHPNVICDGCNGPVVGTRYKCSVCPDYDLCSVCEGKGLHRGHTKLAFPSPFGHLSEGFSHSRWLRKVKHGHFGWPGWEMGPPGNWSPRPPRAGEARPGPTAESASGPSEDPSVNFLKNVGESVAAALSPLGIEVDIDVEHGGKRSRLTPVSPESSSTEEKSSSQPSSCCSDPSKPGGNVEGATQSLAEQMRKIALESEGRPEEQMESDNCSGGDDDWTHLSSKEVDPSTGELQSLQMPESEGPSSLDPSQEGPTGLKEAALYPHLPPEADPRLIESLSQMLSMGFSDEGGWLTRLLQTKNYDIGAALDTIQYSKHPPPL

SEQ ID NO: 17 - protein, 465 aa, Homo sapiens
MIVFVRFNSSHGFPVEVDSDTSIFQLKEVVAKRQGVPADQLRVIFAGKELRNDWTVQNCDLDQQSIVHIVQRPWRKGQEMNATGGDDPRNAAGGCEREPQSLTRVDLSSSVLPGDSVGLAVILHTDSRKDSPPAGSPAGRSIYNSFYVYCKGPCQRVQPGKLRVQCSTCRQATLTLTQGPSCWDDVLIPNRMSGECQSPHCPGTSAEFFFKCGAHPTSDKETSVALHLIATNSRNITCITCTDVRSPVLVFQCNSRHVICLDCFHLYCVTRLNDRQFVHDPQLGYSLPCVAGCPNSLIKELHHFRILGEEQYNRYQQYGAEECVLQMGGVLCPRPGCGAGLLPEPDQRKVTCEGGNGLGCGFAFCRECKEAYHEGECSAVFEASGTTTQAYRVDERAAEQARWEAASKETIKKTTKPCPRCHVPVEKNGGCMHMKCPQPQCRLEWCWNCGCEWNRVCMGDHWFDV

SEQ ID NO: 18 - protein, 194 aa, Homo sapiens
MSQNGAPGMQEESLQGSWVELHFSNNGNGGSVPASVSIYNGDMEKILLDAQHESGRSSSKSSHCDSPPRSQTPQDTNRASETDTHSIGEKNSSQSEEDDIERRKEVESILKKNSDWIWDWSSRPENIPPKEFLFKHPKRTATLSMRNTSVMKKGGIFSAEFLKVFLPSLLLSHLLAIGLGIYIGRRLTTSTSTF

SEQ ID NO: 19 - protein, 283 aa, Homo sapiens
MAVPPTYADLGKSARDVFTKGYGFGLIKLDLKTKSENGLEFTSSGSANTETTKVTGSLETKYRWTEYGLTFTEKWNTDNTLGTEITVEDQLARGLKLTFDSSFSPNTGKKNAKIKTGYKREHINLGCDMDFDIAGPSIRGALVLGYEGWLAGYQMNFETAKSRVTQSNFAVGYKTDEFQLHTNVNDGTEFGGSIYQKVNKKLETAVNLAWTAGNSNTRFGIAAKYQIDPDACFSAKVNNSSLIGLGYTQTLKPGIKLTLSALLDGKNVNAGGHKLGLGLEFQA

SEQ ID NO: 20 - protein, 105 aa, Homo sapiens
MGDVEKGKKIFIMKCSQCHTVEKGGKHKTGPNLHGLFGRKTGQAPGYSYTAANKNKGIIWGEDTLMEYLENPKKYIPGTKMIFVGIKKKEERADLIAYLKKATNE

SEQ ID NO: 21 - protein, 260 aa, Homo sapiens
MKIKNFFLNFNRKSVQNNNSLNHFDLSYRVFRIYRWNPSIMKNPWINSYIVSMNNCGPIVLDALIKIKNEQDSTLTFRRSCREGICGSCAINIIGKNTLACLYKLSDSSRFIHIYPLPHIYVIKDLVPDLTNFYTQHKSIQPWLQPNRDYSLQKEFIQSKQERSYLDGLYECILCACCSASCPSYWWNGDRYLGPAILLQAYRWIIDSRDSATEKRLGYLNDSYKVYRCHTIMNCTKTCPKHLNPGKAIANIKILLKGEK

SEQ ID NO: 22 - protein, 81 aa, Homo sapiens
MLRQIIGQAKKHPSLIPLFVFIGTGATGATLYLLRLALFNPDVCWDRNNPEPWNKLGPNDQYKFYSVNVDYSKLKKERPDF

SEQ ID NO: 23 - protein, 553 aa, Homo sapiens
MLSVRVAAAVVRALPRRAGLVSRNALGSSFIAARNFHASNTHLQKTGTAEMSSILEERILGADTSVDLEETGRVLSIGDGIARVHGLRNVQAEEMVEFSSGLKGMSLNLEPDNVGVVVFGNDKLIKEGDIVKRTGAIVDVPVGEELLGRVVDALGNAIDGKGPIGSKTRRRVGLKAPGIIPRISVREPMQTGIKAVDSLVPIGRGQRELIIGDRQTGKTSIAIDTIINQKRFNDGSDEKKKLYCIYVAIGQKRSTVAQLVKRLTDADAMKYTIVVSATASDAAPLQYLAPYSGCSMGEYFRDNGKHALIIYDDLSKQAVAYRQMSLLLRRPPGREAYPGDVFYLHSRLLERAAKMNDAFGGGSLTALPVIETQAGDVSAYIPTNVISITDGQIFLETELFYKGIRPAINVGLSVSRVGSAAQTRAMKQVAGTMKLELAQYREVAAFAQFGSDLDAATQQLLSRGVRLTELLKQGQYSPMAIEEQVAVIYAGVRGYLDKLEPSKITKFENAFLSHVVSQHQALLGTIRADGKISEQSDAKLKEIVTNFLAGFEA

SEQ ID NO: 24 - protein, 727 aa, Homo sapiens
MLRIPVRKALVGLSKSPKGCVRTTATAASNLIEVFVDGQSVMVEPGTTVLQACEKVGMQIPRFCYHERLSVAGNCRMCLVEIEKAPKVVAACAMPVMKGWNILTNSEKSKKAREGVMEFLLANHPLDCPICDQGGECDLQDQSMMFGNDRSRFLEGKRAVEDKNIGPLVKTIMTRCIQCTRCIRFASEIAGVDDLGTTGRGNDMQVGTYIEKMFMSELSGNIIDICPVGALTSKPYAFTARPWETRKTESIDVMDAVGSNIVVSTRTGEVMRILPRMHEDINEEWISDKTRFAYDGLKRQRLTEPMVRNEKGLLTYTSWEDALSRVAGMLQSFQGKDVAAIAGGLVDAEALVALKDLLNRVDSDTLCTEEVFPTAGAGTDLRSNYLLNTTIAGVEEADVVLLVGTNPRFEAPLFNARIRKSWLHNDLKVALIGSPVDLTYTYDHLGDSPKILQDIASGSHPFSQVLKEAKKPMVVLGSSALQRNDGAAILAAVSSIAQKIRMTSGVTGDWKVMNILHRIASQVAALDLGYKPGVEAIRKNPPKVLFLLGADGGCITRQDLPKDCFIIYQGHHGDVGAPIADVILPGAAYTEKSATYVNTEGRAQQTKVAVTPPGLAREDWKIIRALSEIAGMTLPYDTLDQVRNRLEEVSPNLVRYDDIEGANYFQQANELSKLVNQQLLADPLVPPQLTIKDFYMTDSISRASQTMAKCVKAVTEGAQAVEEPSIC

SEQ ID NO: 25 - protein, 81 aa, Homo sapiens; identical to SEQ ID NO: 22